(19) United States
(12) Patent Application Publication
(10) Pub. No.: US 2009/0118277 A1
(43) Pub. Date: May 7, 2009

(54) 3 Unsubstituted N-(aryl- or heteroaryl)-pyrazolo[1,5-a]pyrimidines as Kinase Inhibitors

(75) Inventors: Keiichi Masuya, Bottmingen (CH); Andrea Vaupel, Riehen (CH); Patricia Imbach, Kaiseraugst (CH); Pascal Furet, Thann (FR)

Correspondence Address: NOVARTIS;CORPORATE INTELLECTUAL PROPERTY, ONE HEALTH PLAZA 104/3, EAST HANOVER, NJ, 07936-1080, US

(73) Assignee: Novartis AG

(21) Appl. No.: 12/296,156

(22) PCT Filed: Apr. 2, 2007
(86) PCT No.: PCT/EP07/02952

§ 371 Date: Oct. 6, 2008

(30) Foreign Application Priority Data
Apr. 4, 2006 (GB) 0606804.3

(51) Int. Cl. A61K 31/519; C07D 487/04; A61K 31/5377; A61P 43/00; C07D 413/14

(52) U.S. Cl. 514/234.2; 544/281; 544/117; 514/259.31

(57) ABSTRACT

The invention relates to 3-unsubstituted N-(aryl- or heteroaryl)-pyrazolo[1,5-a]pyrimidine compounds, their use as kinase inhibitors, new pharmaceutical formulations comprising said compounds, said compounds for use in the diagnostic or therapeutic treatment of warm-blooded animals, especially humans, their use in the treatment of diseases or for the manufacture of pharmaceutical formulations useful in the treatment of diseases that respond to modulation of kinase, especially tie-2 kinase, activity, methods of treatment comprising administration of said compounds to a warm-blooded animal, especially a human, and processes for the manufacture of said compounds.

The invention relates to 3-unsubstituted N-(aryl- or heteroaryl)-pyrazolo[1,5-a]pyrimidine compounds, their use as kinase inhibitors, new pharmaceutical formulations comprising said compounds, said compounds for use in the diagnostic or therapeutic treatment of warm-blooded animals, especially humans, their use in the treatment of diseases or for the manufacture of pharmaceutical formulations useful in the treatment of diseases that respond to modulation of kinase, especially tie-2 kinase, activity, methods of treatment comprising administration of said compounds to a warm-blooded animal, especially a human, and processes for the manufacture of said compounds.

The term kinases comprises both receptor-type kinases and nonreceptor-type kinases, as well as tyrosine and serine/threonine kinases. Among the receptor type tyrosine kinases, Tie-2 (which is also called TEK) is expressed in endothelial cells that line the lumen of blood vessels. It has been shown to be involved in endothelial cell migration, sprouting, survival and periendothelic cell recruitment during angiogenesis.

In contrast to VEGFRs (vascular endothelial growth factor receptors), which control the onset of angiogenesis, the angiopoietins (ligands of Tie-2) and Tie-2 are involved in vessel stabilization and vascular remodeling. It could be shown that Tie-2 is activated by one of its ligands, angiopoietin-1, which is antagonized by a second ligand, angiopoietin-2 (ang2). Where angiogenesis takes place, the antagonist ang2 is up-regulated. Therefore there was hitherto no direct clue allowing to reasonably assume whether inhibition of Tie-2 promotes or inhibits angiogenesis, but this concept has been confirmed in the meantime.

On the other hand, in view of the many possible mechanisms involved in the pathogenesis of tumor and other proliferative diseases, a need exists to find novel and useful modulators of the activity of kinases which often are involved in their genesis. Therefore novel compounds that modulate the activity of other kinases than those already established compounds as useful in the treatment of proliferative diseases and that can affect tumor growth, especially in cases where no effect is found with VEGFR inhibitors, are highly desirable.

It is therefore a problem to be solved by the present invention to provide novel chemical compounds with advantageous pharmaceutical properties that are useful in the treatment of proliferative diseases, such as tumor diseases.

Surprisingly, it is possible to establish that a novel class of 3-substituted N-(aryl- or heteroaryl)pyrazolo[1,5-a]pyrimidine compounds is capable to inhibit the growth of tumors in tumor models that depend on angiogenesis. Especially, it has been found that these compounds can inhibit Tie-2 kinase quite specifically and could be sufficient to inhibit VEGF-induced angiogenesis in vivo when tested, for example, in a subcutaneous growth factor chamber implant model and can show, for example, qualitative differences to VEGFR2 inhibitors.

The invention therefore relates to novel compounds of the formula I,

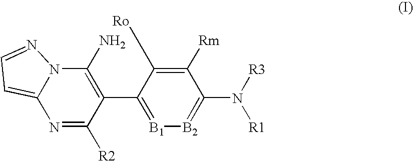

wherein
R1 is acyl,
R2 is hydrogen, lower alkyl, heterocyclyl-lower alkyl wherein heterocyclyl is unsubstituted or substituted and has 3 to 14 ring atoms, hydroxyl-lower alkyl, esterified or etherified hydroxyl-lower alkyl or unsubstituted or substituted amino-lower alkyl;
R3 is hydrogen or unsubstituted or substituted lower alkyl;

### B₁ is N or CRo;

### B₂ is N or CRm;

and each Ro and Rm, independently of the others, is selected from hydrogen, lower alkyl, halo and lower alkoxy;
with the proviso that if R1 is (trifluoromethylphenyl)-aminocarbonyl, then R2 is heterocyclyl-lower alkyl wherein heterocyclyl is unsubstituted or substituted and has 3 to 14 ring atoms, hydroxyl-lower alkyl, esterified or etherified hydroxyl-lower alkyl or unsubstituted or substituted amino-lower alkyl (that is other than hydrogen) and/or R3 is unsubstituted or substituted lower alkyl (that is other than hydrogen);
or a salt thereof.

Listed below are definitions of various terms used to describe the compounds of the present invention as well as their use and synthesis, starting materials and intermediates and the like. These definitions, either by replacing one, more than one or all general expressions or symbols used in the present disclosure and thus yielding preferred embodiments of the invention, preferably apply to the terms as they are used throughout the specification unless they are otherwise limited in specific instances either individually or as part of a larger group. In other terms: Independently of each other, one or more of the more general expressions may be replaced by the more specific definitions, thus leading to preferred embodiments of the invention.

The term “lower” or “C₁-C₇-” defines a moiety with up to and including maximally 7, especially up to and including maximally 4, carbon atoms, said moiety being branched (one or more times) or straight-chained and bound via a terminal or a non-terminal carbon. Lower or C₁-C₇-alkyl, for example, is n-pentyl, n-hexyl or n-heptyl or preferably C₁-C₄-alkyl, especially as methyl, ethyl, n-propyl, sec-propyl, n-butyl, isobutyl, sec-butyl, tert-butyl.

Halo or halogen is preferably fluoro, chloro, bromo or iodo, most preferably fluoro, chloro or bromo.

Acyl is preferably the moiety (remaining after the removal of the acidic hydrogen) of an organic carbonic or sulfonic acid with (without substitutents) 1 to 22 carbon atoms, and is preferably selected from the group consisting of unsubstituted or substituted C₆-C₁₄-aryl-aminocarbonyl (═C₆-C₁₄-aryl-NH—C(═O)—), unsubstituted or substituted heterocyclylaminocarbonyl (=heterocyclyl-N—C(═O)—) wherein heterocyclyl has 3 to 14 ring atoms, unsubstituted or substituted C₆-C₁₄-arylaminosulfonyl (=aryl-NH—S(O)₂—), unsubstituted or substituted heterocyclylaminosulfonyl (=heterocylyl-NH—S(O)₂) wherein heterocyclyl has 3 to 14 ring atoms, unsubstituted or substituted lower-alkanesulfonyl (=lower-alkane-S(O)₂—), unsubstituted or substituted C₆-C₁₄-arylsulfonyl (=aryl-S(O)₂—), unsubstituted or substituted heterocyclylsulfonyl (=heterocyclyl-S(O)₂—) wherein heterocyclyl has 3 to 14 ring atoms, and unsubstituted or substituted C₆-C₁₄-arylcarbonyl (=aryl-C(═O)—);

In unsubstituted or substituted C₆-C₁₄-arylaminocarbonyl, unsubstituted or substituted C₆-C₁₄-aryl is preferably defined as below; more preferred is a moiety selected from phenylaminocarbonyl wherein phenyl is unsubstituted or substituted by one or more, especially up to two, moieties independently selected from lower alkyl, especially methyl, halo (very preferred), especially chloro; halo-lower alkyl, such as trifluoromethyl, lower alkoxy, such as methoxy, and cyano. Very preferred is 3-trifluoromethyl-phenylaminocarbonyl, more preferred 4-fluorophenylaminocarbonyl and most preferred (especially 3- or 2-) chlorophenylaminocarbonyl.

In unsubstituted or substituted heterocyclylaminocarbonyl wherein heterocyclyl has 3 to 14 ring atoms, unsubstituted or substituted heterocyclyl is preferably as defined below; more preferred is pyrazolyl-aminocarbonyl (especially pyrazol-5-ylaminocarbonyl) or isoxazolyl-aminocarbonyl (especially isoxazol-3-ylaminocarbonyl), where each pyrazolyl or isoxazolyl is unsubstituted or substituted by one or two moieties independently selected from the group consisting of lower alkyl, such as tert-butyl, and phenyl that is unsubstituted or substituted with halo, especially fluoro, lower alkoxy, especially methoxy, piperazino-lower alkyl, especially piperazinomethyl, 4-lower alkylpiperazino-lower alkyl, such as 4-methylpiperazino-methyl, and morpholino-lower alkyl, especially morpholinomethyl. Very preferred is 3-tert-butyl-1-(4-fluorophenyl)-pyrazol-5-ylaminocarbonyl, 3-tert-butyl-1-(4-methoxyphenyl)-pyrazol-5-ylaminocarbonyl, 3-tert-butyl-1-(4-(4-methyl-piperazinomethyl)-phenyl)-pyrazol-5-ylaminocarbonyl, 3-tert-butyl-1-(3-(4-methyl-piperazinomethyl)-phenyl)-pyrazol-5-ylaminocarbonyl, 3-tert-butyl-1-(4-(morpholinomethyl)-phenyl)-pyrazol-5-ylaminocarbonyl or 5-tert-butyl-isoxazol-3-ylaminocarbonyl.

In unsubstituted or substituted C₆-C₁₄-arylaminosulfonyl, unsubstituted or substituted C₆-C₁₄-aryl is preferably as described below. Very preferred is 3-trifluoromethyl-phenylaminosulfonyl, more preferred 4-fluorophenylaminosulfonyl and most preferred 3- or 2-chlorophenylaminosulfonyl.

In unsubstituted or substituted heterocyclylaminosulfonyl wherein heterocyclyl has 3 to 14 ring atoms, unsubstituted or substituted heterocylyl is preferably as defined below; more preferred is pyrazolyl-aminosulfonyl (especially pyrazol-5-ylaminosulfonyl) or isoxazolylaminosulfonyl (especially isoxazol-3-ylaminosulfonyl), where each pyrazolyl or isoxazolyl is unsubstituted or substituted by one or two moieties independently selected from the group consisting of lower alkyl, such as tert-butyl, and phenyl that is unsubstituted or substituted with halo, especially fluoro, lower alkoxy, especially methoxy, piperazino-lower alkyl, especially piperazinomethyl, 4-lower alkylpiperazino-lower alkyl, such as 4-methylpiperazino-methyl, and morpholino-lower alkyl, epecially morpholinomethyl. Very preferred is 3-tert-butyl-1-(4-fluorophenyl)-pyrazol-5-ylaminosulfonyl.

In unsubstituted or substituted lower-alkanesulfonyl, unsubstituted or substituted lower alkyl is preferably as defined below; more preferred is phenyl-lower alkanesulfonyl, such as phenylmethylsulfonyl or 2-phenylethylsulfonyl, wherein each phenyl is unsubstituted (preferred) or substituted with one or more, e.g. up to three, moieties independently selected from the group consisting of lower alkyl, e.g. methyl, halo, e.g. chloro or fluoro, halo-lower alkyl, e.g. trifluoromethyl, lower alkoxy, e.g. methoxy, and cyano. Very preferred is phenylmethylsulfonyl or 2-phenylethylsulfonyl.

In unsubstituted or substituted C₆-C₁₄-arylsulfonyl, unsubstituted or substituted C₆-C₁₄-aryl is preferably as defined below; more preferred is phenylsulfonyl wherein the phenyl is unsubstituted or substituted by one or more, e.g. up to three, more preferably up to two, moieties independently selected from the group consisting of lower alkyl, e.g. methyl, halo (preferred), such as chloro (very preferred) or fluoro, halo-lower alkyl, e.g. trifluoromethyl, lower alkoxy, e.g. methoxy; and cyano. Very preferred is 2,3-dimethylphenylsulfonyl, 2-, 3- or 4-methylphenylsulfonyl, 3- or 4-methoxyphenylsulfonyl, 2-methyl-4,5-dimethoxyphenylsulfonyl, 2,5-dimethoxyphenylsulfonyl, 2-, 3- or 4-trifluoromethylphenylsulfonyl, 2-chloro-5-trifluoromethylphenylsulfonyl, 2-chloro-4-trifluoromethylphenylsulfonyl, and especially 2,-3- or 4-chlorophenylsulfonyl, 2,3-, 2,4-, 2,5-, 3,5- or 2,6-dichlorophenylsulfonyl, 2-chloro-4-cyanophenylsulfonyl or 4-fluoro-2-chlorophenylsulfonyl.

In unsubstituted or substituted heterocyclylsulfonyl wherein heterocyclyl has 3 to 14 ring atoms, unsubstituted or substituted heterocyclyl is preferably as described below; more preferred is isoxazolylsulfonyl wherein isoxazolyl is unsubstituted or substituted by one or more, e.g. up to two, independently selected lower alkyl moieties. Very preferred is 5-methyl- or 3,5-dimethyl-isoxazol-4-ylsulfonyl.

In unsubstituted or substituted C₆-C₁₄-arylcarbonyl, unsubstituted or substituted aryl is preferably as defined above; more preferred is benzoyl substituted by one or more, e.g. up to two, independently selected halo moieties, especially chloro. Very preferred is 2- or 3-chlorobenzoyl.

In heterocyclyl-lower alkyl wherein heterocyclyl is unsubstituted or substituted and has 3 to 14 ring atoms, unsubstituted or substituted heterocylyl is preferably as defined below and is attached to linear or branched lower alkyl, especially at a terminal carbon atom, e.g. to methyl; more preferred is pyrrolidino-, piperidinyl-, such as piperidino, piperazino or lower-alkyl-piperazino, such as 4-lower alkylpiperazino.

In (unsubstituted or substituted amino)-lower alkoxy, amino is preferably unsubstituted or N-mono- or N,N-di-substituted by unsubstituted or substituted lower alkyl as defined below and the lower alkyl in lower alkoxy is linear or branched, preferably linear with the (unsubstituted or substituted amino) at the terminal carbon atom; more preferred is N-mono- or N,N-di-(lower alkyl and/or phenyl-lower alkyl)amino-lower alkoxy or amino-lower alkoxy, especially 2-amino-ethyl or 3-aminopropyl.

Unsubstituted or substituted lower alkyl is preferably (linear or branched) lower alkyl that is unsubstituted or substituted by one or more, e.g. one to three, substituents, e.g. at a terminal carbon atom, independently selected from the group consisting of unsubstituted or substituted C₆-C₁₄-aryl as described below, especially phenyl or naphthyl, (each of) which is unsubstituted or substituted as described below for unsubstituted or substituted C₆-C₁₄-aryl, unsubstituted or substituted heterocyclyl with 3 to 14 ring atoms as described below which is unsubstituted or substituted as described below for unsubstituted or substituted heterocyclyl, especially piperidino, morpholino, thiomorpholino, N—C₁-C₇-alkyl-piperazino, pyridyl, e.g. pyridine-2-yl or pyridine-3-yl, or N-mono- or N,N-di-(C₁-C₇-alkyl-substituted or unsubstituted pyrrolidino, unsubstituted or substituted cycloalkyl as described below, especially cyclopropyl, cyclobutyl, cyclopentyl or cyclohexyl each of which is unsubstituted or substituted as described below for unsubstituted or substituted cycloalkyl, halo, e.g. in trifluoromethyl, hydroxy, halo-C₁-C₇-alkoxy, such as trifluoromethoxy, hydroxy-C₁-C₇-alkoxy, C₁-C₇-alkoxy-C₁-C₇-alkoxy, phenyl- or naphthyloxy, phenyl- or naphthyl-C₁-C₇-alkyloxy, C₁-C₇-alkanoyloxy, benzoyl- or naphthoyloxy, C₁-C₇-alkylthio, halo-C₁-C₇-alkthio, such as trifluoromethylthio, C₁-C₇-alkoxy-C₁-C₇-alkylthio, phenyl- or naphthylthio, phenyl- or naphthyl-C₁-C₇-alkylthio, C₁-C₇-alkanoylthio, benzoyl- or naphthoylthio, nitro, amino, mono- or di-(C₁-C₇-alkyl, C₁-C₇-alkoxy-C₁-C₇alkyl and/or (mono- or di-(C₁-C₇-alkyl)-amino)-C₁-C₇-alkyl)-amino, mono- or di-(naphthyl- or phenyl-C₁-C₇-alkyl)-amino, C₁-C₇-alkanoylamino, benzoyl- or naphthoylamino, C₁-C₇-alkylsulfonylamino, phenyl- or naphthylsulfonylamino wherein phenyl or naphthyl is unsubstituted or substituted by one or more, especially one to three, C₁-C₇-alkyl moieties, phenyl- or naphthyl-C₁-C₇-alkylsulfonylamino, carboxyl, C₁-C₇-alkyl-carbonyl, C₁-C₇-alkoxy-carbonyl, phenyl- or naphthyloxycarbonyl, phenyl- or naphthyl-C₁-C₇-alkoxycarbonyl, carbamoyl, N-mono- or N,N-di-(C₁-C₇-alkyl)-aminocarbonyl, N-mono- or N,N-di-(naphthyl- or phenyl-C₁-C₇-alkyl)-aminocarbonyl, cyano, C₁-C₇-alkenylene or -alkynylene, C₁-C₇-alkylenedioxy, C₁-C₇-alkylsulfonyl, phenyl- or naphthylsulfonyl wherein phenyl or naphthyl is unsubstituted or substituted by one or more, especially one to three, C₁-C₇-alkyl moieties, phenyl- or naphthyl-C₁-C₇-alkylsulfonyl, sulfamoyl and N-mono or N,N-di-(C₁-C₇-alkyl, phenyl, naphthyl, phenyl-C₁-C₇-alkyl or naphthyl-C₁-C₇-alkyl)-aminosulfonyl.

Unsubstituted or substituted C₃-C₁₀-cycloalkyl is preferably cyclopropyl, cyclobutyl, cyclopentyl or cyclohexyl and is substituted or (preferably) unsubstituted by one or more substitutents as mentioned for substituted lower alkyl (other than unsubstituted or substituted cycloalkyl).

Unsubstituted or substituted C₆-C₁₄-aryl preferably is a mono- or polycyclic, especially monocyclic, bicyclic or tricyclic aryl moiety with 6 to 14 ring carbon atoms, especially phenyl (very preferred), naphthyl (preferred), indenyl, fluorenyl, acenapthylenyl, phenylenyl or phenanthryl, and is unsubstituted or substituted by one or more, especially one to three, moieties, preferably independently selected from the group consisting of C₁-C₇-alkyl, such as methyl, ethyl, n-propyl, isopropyl, n-butyl, isobutyl, sec-butyl or tert-butyl, C₂-C₇-alkenyl, C₂-C₇-alkinyl, phenyl- or naphthyl-C₁-C₇-alkyl, such as benzyl or naphthylmethyl, halo-C₁-C₇-alkyl, such as trifluoromethyl, hydroxy-C₁-C₇-alkyl, C₁-C₇-alkoxy-C₁-C₇-alkyl, such as 3-methoxypropyl or 2-methoxyethyl, C₁-C₇-alkoxy-C₁-C₇-alkoxy-C₁-C₇-alkyl, phenyloxy- or naphthyloxy-C₁-C₇-alkyl, phenyl-C₁-C₇-alkoxy- or naphthyl-C₁-C₇-alkoxy-C₁-C₇-alkyl, amino-C₁-C₇-alkyl, such as aminomethyl, N-mono- or N,N-di-(C₁-C₇-alkyl and/or mono-C₁-C₇-alkoxy-C₁-C₇alkyl and/or (mono- or di-(C₁-C₇-alkyl)-amino-C₁-C₇-alkyl)-amino-C₁-C₇-alkyl, C₁-C₇-alkoxy-C₁-C₇-alkylamino-C₁-C₇-alkyl, mono- or di-(naphthyl- or phenyl-C₁-C₇-alkyl)-amino-C₁-C₇-alkyl, C₁-C₇-alkanoylamino-C₁-C₇-alkyl, carboxy-C₁-C₇-alkyl, benzoyl- or naphthoylamino-C₁-C₇-alkyl, C₁-C₇-alkylsulfonylamino-C₁-C₇-alkyl, phenyl- or naphthylsulfonylamino-C₁-C₇-alkyl wherein phenyl or naphthyl is unsubstituted or substituted by one or more, especially one to three, C₁-C₇-alkyl moieties, phenyl- or naphthyl-C₁-C₇-alkylsulfonylamino-C₁-C₇-alkyl, pyrrolidino-C₁-C₇-alkyl, piperidino-C₁-C₇-alkyl, morpholino-C₁-C₇-alkyl, thiomorpholino-C₁-C₇-alkyl, N—C₁-C₇-alkyl-piperazino-C₁-C₇-alkyl, N-mono- or N,N-di-(C₁-C₇-alkyl)-amino-substituted or unsubstituted pyrrolidino-C₁-C₇-alkyl, halo (which is especially preferred as substitutent in aryl moieties forming part of R1), especially fluoro, chloro (which is especially preferred) or bromo, hydroxy, C₁-C₇-alkoxy, phenyl-C₁-C₇-alkoxy wherein phenyl is unsubstituted or substituted by C₁-C₇-alkoxy and/or halo, halo-C₁-C₇-alkoxy, such as trifluoromethoxy, hydroxy-C₁-C₇-alkoxy, C₁-C₇-alkoxy-C₁-C₇-alkoxy, amino-C₁-C₇-alkoxy, N—C₁-C₇-alkanoylamino-C₁-C₇-alkoxy, N-unsubstituted-, N-mono- or N,N-di-(C₁-C₇-alkyl)carbamoyl-C₁-C₇-alkoxy, phenyl- or naphthyloxy, phenyl- or naphthyl-C₁-C₇-alkyloxy, C₁-C₇-alkanoyloxy, benzoyl- or naphthoyloxy, C₁-C₇-alkylthio, halo-C₁-C₇-alkthio, such as trifluoromethylthio, C₁-C₇-alkoxy-C₁-C₇-alkylthio, phenyl- or naphthylthio, phenyl- or naphthyl-C₁-C₇-alkylthio, C₁-C₇-alkanoylthio, benzoyl- or naphthaylthio, nitro, amino, mono- or di-(C₁-C₇-alkyl)-amino, mono- or di-(naphthyl- or phenyl-C₁-C₇-alkyl)-amino, C₁-C₇-alkanoylamino, benzoyl- or naphthoylamino, C₁-C₇-alkylsulfonylamino, phenyl- or naphthylsulfonylamino wherein phenyl or naphthyl is unsubstituted or substituted by one or more, especially one to three, C₁-C₇-alkyl moieties, phenyl- or naphthyl-C₁-C₇-alkylsulfonylamino, C₁-C₇-alkanoyl, C₁-C₇-alkoxy-C₁-C₇-alkanoyl, carboxyl, C₁-C₇-alkyl-carbonyl, C₁-C₇-alkoxy-carbonyl, phenyl- or naphthyloxycarbonyl, phenyl- or naphthyl-C₁-C₇-alkoxycarbonyl, carbamoyl, N-mono- or N,N-di-(C₁-C₇-alkyl and/or mono-C₁-C₇-alkoxy-C₁-C₇-alkyl and/or (mono- or di-(C₁-C₇-alkyl)-amino-C₁-C₇-alkyl)-amino-carbonyl, such as N-mono- or N,N-di-(C₁-C₇-alkyl)-aminocarbonyl, N—C₁-C₇-alkoxy-C₁-C₇-alkylcarbamoyl, N-mono- or N,N-di-(naphthyl- or phenyl-C₁-C₇-alkyl)-aminocarbonyl, pyrrolidinocarbonyl, piperidinocarbonyl, morpholinocarbonyl, thiomorpholinocarbonyl, N—C₁-C₇-alkyl-piperazinocarbonyl, N-mono or N,N-di-(C₁-C₇-alkyl)-amino-substituted or unsubstituted pyrrolidino-C₁-C₇-alkyl, cyano, C₁-C₇-alkenylene or -alkinylene, C₁-C₇-alkylsulfonyl, phenyl- or naphthylsulfonyl wherein phenyl or naphthyl is unsubstituted or substituted by one or more, especially one to three, C₁-C₇-alkyl moieties, phenyl- or naphthyl-C₁-C₇-alkylsulfonyl, sulfamoyl and N-mono or N,N-di-(C₁-C₇-alkyl, phenyl-, naphthyl-, phenyl-C₁-C₇-alkyl- or naphthyl-C₁-C₇-alkyl)-aminosulfonyl, piperidino, morpholino, thiomorpholino, N—C₁-C₇-alkyl-piperazino, or N-mono- or N,N-di-(C₁-C₇-alkyl)-amino-substituted or unsubstituted pyrrolidino. Especially preferably aryl is phenyl or naphthyl, each of which is unsubstituted or substituted by one or more, e.g. up to three, substituents independently selected from the group consisting of C₁-C₇-alkyl, hydroxy-C₁-C₇-alkyl, C₁-C₇-alkoxy-C₁-C₇-alkyl, halo-C₁-C₇-alkyl, pyrrolidino-C₁-C₇-alkyl, piperidino-C₁-C₇-alkyl, morpholino-C₁-C₇-alkyl, thiomorpholino-C₁-C₇-alkyl, N—C₁-C₇-alkyl-piperazino-C₁-C₇-alkyl, N-mono- or N,N-di-(C₁-C₇-alkyl)-amino-substituted or unsubstituted pyrrolidino-C₁-C₇-alkyl, halo, especially fluoro, chloro or bromo, hydroxy, C₁-C₇-alkoxy, C₁-C₇-alkoxy-C₁-C₇-alkoxy, amino-C₁-C₇-alkoxy, N—C₁-C₇-alkanoylamino-C₁-C₇-alkoxy, carbamoyl-C₁-C₇-alkoxy, N-mono- or N,N-di-(C₁-C₇-alkyl)-carbamoyl-C₁-C₇-alkoxy, amino, C₁-C₇-alkanoylamino, C₁-C₇-alkanoyl, C₁-C₇-alkoxy-C₁-C₇-alkanoyl, carboxy, C₁-C₇-alkoxycarbonyl, carbamoyl, N-mono- or N,N-di-(C₁-C₇-alkyl and/or C₁-C₇-alkoxy-C₁-C₇-alkyl)-carbamoyl, pyrrolidinocarbonyl, piperidinocarbonyl, morpholinocarbonyl, thiomorpholinocarbonyl, N—C₁-C₇-alkyl-piperazinocarbonyl, N-mono- or N,N-di-(C₁-C₇-alkyl)-amino-substituted or unsubstituted pyrrolidino-C₁-C₇-alkyl, nitro, cyano, pyrrolidino, piperidino, morpholino, thiomorpholino, N—C₁-C₇-alkyl-piperazino, and N-mono- or N,N-di-(C₁-C₇-alkyl)-amino-substituted or unsubstituted pyrrolidino.

Unsubstituted or substituted heterocyclyl with 3 to 14 ring atoms is preferably a heterocyclic radical that is unsaturated, saturated or partially saturated in the bonding ring and is preferably a monocyclic or in a broader aspect of the invention poly-, e.g. bi- or tri-cyclic ring; has 3 to 14 ring atoms; wherein at least in the ring bonding to the remaining part of the molecule of formula I one or more, preferably one to four, especially one or two carbon ring atoms are replaced by a heteroatom selected from the group consisting of nitrogen, oxygen and sulfur, the bonding ring preferably having 4 to 12, especially 5 to 7 ring atoms; heterocyclyl being unsubstituted or substituted by one or more, especially 1 to 3, substituents independently selected from the group consisting of the substituents defined above under “substituted alkyl” or “substituted aryl”; especially being a heterocyclyl radical selected from the group consisting of oxiranyl, azirinyl, 1,2-oxathiolanyl, imidazolyl, thienyl, furyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl (preferred), e.g. pyrrolidino, imidazolyl, imidazolidinyl, benzimidazolyl, pyrazolyl (preferred), pyrazinyl, pyrazolidinyl, pyranyol, thiazolyl, isothiazolyl, dithiazolyl, oxazolyl, isoxazolyl (preferred), e.g. isoxazol-3-yl, (R₅), pyridyl (preferred), e.g. pyridine-2- or -3-yl, pyrazinyl, pyrimidinyl, piperidyl (preferred), e.g. piperidino or piperidin-4-yl, piperazinyl (preferred), e.g. piperazino, pyridazinyl, morpholinyl, thiomorpholinyl, indolizinyl, isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, beta-carbolinyl, phenanthridinyl, acridinyl, perimidinyl, phenanthrolinyl, furazanyl, phenazinyl, phenothiazinyl, phenoxazinyl, chromenyl, isochromanyl and chromanyl, each of these radicals being unsubstituted or substituted by one or more, e.g. one to three radicals independently selected from the group consisting of C₁-C₇-alkyl (preferred), such as methyl, ethyl, n-propyl, isopropyl, n-butyl, isobutyl, sec-butyl or tert-butyl, phenyl (preferred) or naphthyl (preferred) each of which is unsubstituted or substituted by one or more substituents selected from the group consisting of halo, lower alkoxy, pyrrolidinyl-lower alkyl (especially-methyl), piperidinyl-lower alkyl (especially-methyl), piperazino-lower alkyl (especially-methyl), N-lower alkylpiperazino-lower alkyl (especially-methyl), morpholino-lower alkyl (especially-methyl) and thiomorpholino-lower alkyl (especially-methyl), C₂-C₇-alkenyl, C₂-C₇-alkinyl, phenyl- or naphthyl-C₁-C₇-alkyl, such as benzyl or naphthylmethyl, halo-C₁-C₇-alkyl, such as trifluoromethyl, hydroxy-C₁-C₇-alkyl, C₁-C₇-alkoxy-C₁-C₇-alkyl, such as 3-methoxypropyl or 2-methoxyethyl, C₁-C₇-alkoxy-C₁-C₇-alkoxy-C₁-C₇-alkyl, phenyloxy- or naphthyloxy-C₁-C₇-alkyl, phenyl-C₁-C₇-alkoxy- or naphthyl-C₁-C₇-alkoxy-C₁-C₇-alkyl, amino-C₁-C₇-alkyl, such as aminomethyl, N-mono- or N,N-di-(C₁-C₇-alkyl, mono-C₁-C₇-alkoxy-C₁-C₇alkyl and/or (mono- or di-(C₁-C₇-alkyl)-amino-C₁-C₇-alkyl)-amino-C₁-C₇-alkyl, C₁-C₇-alkoxy-C₁-C₇-alkylamino-C₁-C₇-alkyl, mono- or di-(naphthyl- or phenyl-C₁-C₇-alkyl)-amino-C₁-C₇-alkyl, C₁-C₇-alkanoylamino-C₁-C₇-alkyl, carboxy-C₁-C₇-alkyl, benzoyl- or naphthoylamino-C₁-C₇-alkyl, C₁-C₇-alkylsulfonylamino-C₁-C₇-alkyl, phenyl- or naphthylsulfonylamino-C₁-C₇-alkyl wherein phenyl or naphthyl is unsubstituted or substituted by one or more, especially one to three, C₁-C₇-alkyl moieties, phenyl- or naphthyl-C₁-C₇-alkylsulfonylamino-C₁-C₇-alkyl, pyrrolidino-C₁-C₇-alkyl, piperidino-C₁-C₇-alkyl, morpholino-C₁-C₇-alkyl, thiomorpholino-C₁-C₇-alkyl, N—C₁-C₇-alkyl-piperazino-C₁-C₇-alkyl, N-mono- or N,N-di-(C₁-C₇-alkyl)-amino-substituted or unsubstituted pyrrolidino-C₁-C₇-alkyl, halo, especially fluoro, chloro or bromo, hydroxy, C₁-C₇-alkoxy, phenyl-C₁-C₇-alkoxy wherein phenyl is unsubstituted or substituted by C₁-C₇-alkoxy and/or halo, halo-C₁-C₇-alkoxy, such as trifluoromethoxy, hydroxy-C₁-C₇-alkoxy, C₁-C₇-alkoxy-C₁-C₇-alkoxy, amino-C₁-C₇-alkoxy, N—C₁-C₇-alkanoylamino-C₁-C₇-alkoxy, N-unsubstituted-, N-mono- or N,N-di-(C₁-C₇-alkyl)carbamoyl-C₁-C₇-alkoxy, phenyl- or naphthyloxy, phenyl or naphthyl-C₁-C₇-alkyloxy, C₁-C₇-alkanoyloxy, benzoyl- or naphthoyloxy, C₁-C₇-alkylthio, halo-C₁-C₇-alkthio, such as trifluoromethylthio, C₁-C₇-alkoxy-C₁-C₇-alkylthio, phenyl- or naphthylthio, phenyl- or naphthyl-C₁-C₇-alkylthio, C₁-C₇-alkanoylthio, benzoyl- or naphthoylthio, nitro, amino, mono- or di-(C₁-C₇-alkyl)-amino (preferred), mono- or di-(naphthyl- or phenyl-C₁-C₇-alkyl)-amino, C₁-C₇-alkanoylamino, benzoyl- or naphthoylamino, C₁-C₇-alkylsulfonylamino, phenyl- or naphthylsulfonylamino wherein phenyl or naphthyl is unsubstituted or substituted by one or more, especially one to three, C₁-C₇-alkyl moieties, phenyl- or naphthyl-C₁-C₇-alkylsulfonylamino, C₁-C₇-alkanoyl, C₁-C₇-alkoxy-C₁-C₇-alkanoyl, carboxyl, C₁-C₇-alkyl-carbonyl, C₁-C₇-alkoxy-carbonyl, phenyl- or naphthyloxycarbonyl, phenyl- or naphthyl-C₁-C₇-alkoxycarbonyl, carbamoyl, N-mono- or N,N-di-(C₁-C₇-alkyl and/or mono-C₁-C₇-alkoxy-C₁-C₇alkyl and/or (mono- or di-(C₁-C₇-alkyl)-amino-C₁-C₇-alkyl)-amino-carbonyl, such as N-mono- or N,N-di-(C₁-C₇-alkyl)-aminocarbonyl, N—C₁-C₇-alkoxy-C₁-C₇-alkylcarbamoyl, N-mono- or N,N-di-(naphthyl- or phenyl-C₁-C₇-alkyl)-aminocarbonyl, pyrrolidinocarbonyl, piperidinocarbonyl, morpholinocarbonyl, thiomorpholinocarbonyl, N—C₁-C₇-alkyl-piperazinocarbonyl, N-mono- or N,N-di-(C₁-C₇-alkyl)-amino-substituted or unsubstituted pyrrolidino-C₁-C₇-alkyl, cyano, C₁-C₇-alkenylene or -alkinylene, C₁-C₇-alkylsulfonyl (=lower alkanesulfonyl) (preferred), phenyl- or naphthylsulfonyl wherein phenyl or naphthyl is unsubstituted or substituted by one or more, especially one to three, C₁-C₇-alkyl moieties, phenyl- or naphthyl-C₁-C₇-alkylsulfonyl, sulfamoyl and N-mono or N,N-di-(C₁-C₇-alkyl, and phenyl-, naphthyl-, phenyl-C₁-C₇-alkyl- or naphthyl-C₁-C₇-alkyl)-aminosulfonyl.

In heterocyclyl-lower alkyl R2 wherein heterocyclyl is unsubstituted or substituted and has 3 to 14 ring atoms, unsubstituted or substituted heterocyclyl is preferably as defined above and the -lower alkyl is preferably -methyl. More preferred is piperazinolower alkyl, especially piperazinomethyl, 4-lower alkyl-piperazino-lower alkyl, especially 4-methyl-piperazino-methyl.

Hydroxyl-lower alkyl is preferably hydroxyl-methyl.

In esterified hydroxyl-lower alkyl, esterified hydroxyl is preferably acyloxy with acyl as defined above, especially as defined as preferred above, and -lower alkyl is preferably -methyl. Examples are lower alkanoyloxymethyl or benzoxyloxymethyl.

In etherified hydroxyl-lower alkyl, -lower alkyl is preferably -methyl and etherified hydroxyl is preferably
unsubstituted or substituted lower alkyloxy (a preferred substitutent) with unsubstituted or substituted lower alkyl as defined above; more especially lower alkoxy, such as methoxy, hydroxyl-lower alkoxy, such as 2-hydroxy-ethoxy, lower alkoxy-lower alkoxy, such as 2-methoxyethoxy, lower-alkoxy-lower-alkoxy-lower alkoxy, such as 2-(2-(methoxy)-ethoxy)ethoxy, phenyl- or naphthyloxy, or phenyl- or naphthyl-lower alkoxy;
unsubstituted or substituted C₃-C₁₀-cycloalkyloxy wherein unsubstituted or substituted C₃-C₁₀-cycloalkyl is preferably as defined above;
unsubstituted or substituted C₆-C₁₄-aryloxy wherein unsubstituted or substituted C₆-C₁₄-aryl is preferably as defined above; or
unsubstituted or substituted heterocyclyloxy with heterocyclyl with 3 to 14 ring atoms wherein unsubstituted or substituted heterocyclyl with 3 to 14 ring atoms is preferably as defined above.

In unsubstituted or substituted amino-lower alkyl, -lower alkyl is preferably -methyl and unsubstituted or substituted amino is preferably N-mono- or N,N-di-(unsubstituted or substituted alkyl and/or acyl)amino, wherein preferably not more than one acyl moiety is present, wherein unsubstituted or substituted alkyl is preferably as defined above (especially as defined as preferred there), more preferably unsubstituted lower alkyl, and acyl is also preferably as above (especially as defined as preferred there), preferably lower alkanoyl. More preferred is aminomethyl or N-mono- or N,N-di-(lower alkyl and/or phenyl-lower alkyl)aminomethyl.

R2 is preferably hydrogen, lower alkyl, especially methyl, hydroxyl-lower alkyl, especially hydroxylmethyl, lower alkoxy-lower alkyl, especially lower-alkoxymethyl, or (phenyl or naphthyl)-lower alkoxy-lower alkyl, such as (phenyl or naphthyl)-methoxymethyl.

R3 is preferably hydrogen, methyl, piperazinomethyl, 4-methylpiperazinomethyl, 2-aminoethoxy or 3-aminopropoxy.

B₁ is preferably N or Ro, B₂ is preferably CRm.

Preferably, Ro or Rm are hydrogen or one is fluoro, chloro, methyl or methoxy and the other are hydrogen.

Ro and Rm lower alkyl is preferably methyl, halo (which is especially preferred) is especially chloro (very preferred) or fluoro, and lower alkoxy is preferably methoxy.

Salts are especially the pharmaceutically acceptable salts of compounds of formula I. They can be formed where salt forming groups, such as basic or acidic groups, are present that can exist in dissociated form at least partially, e.g. in a pH range from 4 to 10 in aqueous environment, or can be isolated especially in solid form.

Such salts are formed, for example, as acid addition salts, preferably with organic or inorganic acids, from compounds of formula I with a basic nitrogen atom, especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, lactic acid, fumaric acid, succinic acid, citric acid, amino acids, such as glutamic acid or aspartic acid, maleic acid, hydroxymaleic acid, methylmaleic acid, benzoic acid, methane- or ethane-sulfonic acid, ethane-1,2-disulfonic acid, benzenesulfonic acid, 2-naphthalenesulfonic acid, 1,5-naphthalene-disulfonic acid, N-cyclohexylsulfamic acid, N-methyl-, N-ethyl- or N-propyl-sulfamic acid, or other organic protonic acids, such as ascorbic acid.

In the presence of negatively charged radicals, such as carboxy or sulfo, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-dimethylpiperazine.

When a basic group and an acid group are present in the same molecule, a compound of formula I may also form internal salts.

For isolation or purification purposes it is also possible to use pharmaceutically unacceptable salts, for example picrates or perchlorates. For therapeutic use, only pharmaceutically acceptable salts or free compounds are employed (where applicable comprised in pharmaceutical preparations), and these are therefore preferred.

In view of the close relationship between the compounds in free form and in the form of their salts, including those salts that can be used as intermediates, for example in the purification or identification of the compounds or salts thereof, any reference to “compounds” (including also starting materials and “intermediates”) hereinbefore and hereinafter, especially to the compound(s) of the formula I, is to be understood as referring also to one or more salts thereof or a mixture of a free compound and one or more salts thereof, each of which is intended to include also any solvate, metabolic precursor such as ester or amide of the compound of formula I, or salt of any one or more of these, as appropriate and expedient and if not explicitly mentioned otherwise. Different crystal forms may be obtainable and then are also included.

Where the plural form is used for compounds, salts, pharmaceutical preparations, diseases, disorders and the like, this is intended to mean also a single compound, salt, pharmaceutical preparation, disease or the like, and vice versa.

In some cases, a compound of the present invention comprises one or more chiral centers or show other asymmetry (leading to enantiomers) or may otherwise be able to exist in the form of more than one stereoisomer, e.g. due more than one chiral centers or more than one asymmetry or due to rings or double bonds that allow for Z/E (or cis-trans) isomerism (diastereomers). The present inventions includes both mixtures of two or more such isomers, such as mixtures of enantiomers, especially racemates, as well as preferably purified isomers, especially purified enantiomers or enantiomerically enriched mixtures.

The compounds of formula I have valuable pharmacological properties and are useful in the treatment of kinase, especially Tie-2, dependent diseases, e.g., as drugs to treat one or more proliferative diseases.

The terms “treatment” or “therapy” (especially of tyrosine protein kinase dependent diseases or disorders) refer to the prophylactic or preferably therapeutic (including but not limited to palliative, curing, symptom-alleviating, symptom-reducing, kinase-regulating and/or kinase-inhibiting) treatment of said diseases, especially of the diseases mentioned below.

A warm-blooded animal (or patient) is preferably a mammal, especially a human.

Where subsequently or above the term “use” is mentioned (as verb or noun) (relating to the use of a compound of the formula I or a pharmaceutically acceptable salt thereof, this (if not indicated differently or suggested differently by the context) includes any one or more of the following embodiments of the invention, respectively (if not stated otherwise): the use in the treatment of a protein (especially tyrosine, more especially Tie-2) kinase dependent disease, the use for the manufacture of pharmaceutical compositions for use in the treatment of a protein kinase dependent disease, methods of use of one or more compounds of the formula I in the treatment of a protein kinase dependent and/or proliferative disease, pharmaceutical preparations comprising one or more compounds of the formula I for the treatment of said protein kinase dependent disease, and one or more compounds of the formula I in the treatment of said protein kinase dependent disease, as appropriate and expedient, if not stated otherwise. In particular, diseases to be treated and are thus preferred for “use” of a compound of formula I are selected from (especially tyrosine) protein kinase dependent (“dependent” meaning also “supported”, not only “solely dependent”) diseases mentioned below, especially proliferative diseases mentioned below, more especially any one or more of these or other diseases that depend on Tie-2, e.g. aberrantly highly-expressed, constitutively activated, normal and/or mutated Tie-2 kinase.

The (especially important and preferred) efficacy of compounds of the formula I as inhibitors of Tie-2 kinase can be demonstrated as follows:

### Tie-2 Receptor Autophosphorylation

The inhibition of Tie-2 receptor autophosphorylation can be confirmed with an in vitro experiment in cells such as transfected COS cells (ATCC Number: CRL-1651), which permanently express human Tie-2 (SwissProt AccNo Q02763), are seeded in complete culture medium (with 10% fetal calf serum=FCS) in 6-well cell-culture plates and incubated at 37° C. under 5% CO₂ until they show about 90% confluency. The compounds to be tested are then diluted in culture medium (without FCS, with 0.1% bovine serum albumin) and added to the cells. Controls comprise medium without test compounds. After 40 min of incubation at 37° C., ortho vanadate is added to give the final concentration of 10 mM. After a further incubation for 20 minutes at 37° C., the cells are washed twice with ice-cold PBS (phosphate-buffered saline) and immediately lysed in 100 μl lysis buffer per well. The lysates are then centrifuged to remove the cell nuclei, and the protein concentrations of the supernatants are determined using a commercial protein assay (BIORAD). The lysates can then either be immediately used or, if necessary, stored at −20° C.

A sandwich ELISA is carried out to measure the Tie-2 phosphorylation: a monoclonal anti-body to Tie-2 (for example anti-Tie2 clone AB33, Upstate, Cat Nr. 05-584 or comparable monoclonal antibody) is immobilized using 0.1 ml of a 2 μg/ml solution on black ELISA plates (OptiPlate™ HTRF-96 from Packard). The plates are then washed and the remaining free protein-binding sites are saturated with 3% TopBlock® (Juro, Cat. # TB232010) in phosphate buffered saline with Tween 20® (polyoxyethylen(20)sorbitane monolaurate, ICI/Uniquema) (PBST). The cell lysates (100 μg protein per well) are then incubated in these plates overnight at 4° C. together with an antiphosphotyrosine antibody coupled with alkaline phosphatase (PY20:AP from Zymed). The (plates are washed again and the) binding of the anti-phosphotyrosine antibody to the captured phosphorylated receptor is then demonstrated using a luminescent AP substrate (CDP-Star, ready to use, with Emerald II; Applied Biosystems). The luminescence is measured in a Packard Top Count Microplate Scintillation Counter. The difference between the signal of the positive control (stimulated with vanadate) and that of the negative control (not stimulated) corresponds to maximum Tie-2 phosphorylation (=100%). The activity of the tested substances is calculated as percent inhibition of maximum Tie-2 phosphorylation, and the concentration of substance that induces half the maximum inhibition is defined as the IC₅₀ (inhibitory dose for 50% inhibition). For compounds of the formula I, preferably IC₅₀ values in the range from 0.0005 to 5 μM can be found, e.g. more preferably from 0.001 to 1 μM.

For example, of the compounds mentioned in the examples some are especially preferred which show advantageous properties, e.g. the compounds of Example 4, 9, 48 or 49, 5, 8, 16, 17, 18, 19, 27, 39, 30, 52 or 53, or pharmaceutically acceptable salts thereof.

### KDR Autophosphorylation

The activity of the compounds of the invention as inhibitors of KDR protein-tyrosine kinase activity can be tested as follows: The inhibition of VEGF-induced receptor autophosphorylation can be confirmed in cells such as transfected CHO cells, which permanently express human VEGF-R2 receptor (KDR), and are seeded in complete culture medium (with 10% fetal calf serum=FCS) in 6-well cell-culture plates and incubated at 37° C. under 5% CO₂ until they show about 80% confluency. The compounds to be tested are then diluted in culture medium (without FCS, with 0.1% bovine serum albumin) and added to the cells. Controls comprise medium without test compounds. After 2 h incubation at 37° C., recombinant VEGF is added; the final VEGF concentration is 20 ng/ml. After a further incubation period of five minutes at 37° C., the cells are washed twice with ice-cold PBS (phosphate-buffered saline) and immediately lysed in 100 μl lysis buffer per well. The lysates are then centrifuged to remove the cell nuclei, and the protein concentrations of the supernatants are determined using a commercial protein assay (BIORAD). The lysates can then either be immediately used or, if necessary, stored at −20° C. With this assay it can be shown that the compounds of the present invention can show IC₅₀ values for inhibition that are higher (less inhibition) than in the Tie-2 assay. Especially compounds of the formula I wherein R5 is an unsubstituted or substituted (aryl, heterocyclyl or alkane)sulfonyl are selective for Tie-2, while other compounds of the formula I may also be useful as dual inhibitors for both KDR and Tie-2.

A good selectivity can also be found using in vitro assays known in the art against one or more kinases selected from the group consisting of CDK1; IGF-R, insulin receptor kinase, Eph-B4, Raf (e.g. b- and/or c-Raf), Flt-3, Her-1 and FGF-R3. Test systems for many of these are known in the art, see e.g. WO 2005/070431.

The results indicate an advantageous selectivity profile of compounds of the formula I with a quite specific inhibition for Tie-2 kinase, where selectivity does not necessarily mean that only Tie-2 kinase is inhibited to an advantageous and pharmaceutically relevant extent instead also other kinases, e.g. c-Abl, Bcr-Abl, c-Kit, c-Raf, Flt-1, Flt-3, KDR, Her-1, PDGFR-kinase, c-Src, RET-receptor kinase, FGF-R1, FGF-R2, FGF-R3, FGF-R4, Ephrin receptor kinases (e.g., EphB2 kinase, EphB4 kinase and related Eph kinases), casein kinases (CK-1, CK-2, G-CK), Pak, ALK, ZAP70, Jak1, Jak2, AxI, Cdk1, cdk4, cdk5, Met, FAK, Pyk2, Syk, Insulin receptor kinase, or (especially constitutively activating) mutations of kinases (activeting kinases) such as of Bcr-Abl, c-Kit, c-Raf, Flt-3, FGF-R3, PDGF-receptors, RET, and Met, may also be inhibited to an extent to support usefulness in connection with the Tie-2 inhibition.

The efficiency of the compounds of the formula I as inhibitors of tumor growth can be demonstrated as follows:

For example, in order to test whether a compound of the formula I inhibits VEGF-mediated angiogenesis in vivo, its effect on the angiogenic response induced by VEGF in a growth factor implant model in mice can be tested: A porous Teflon chamber (volume 0.5 mL) is filled with 0.8% w/v agar containing heparin (20 units/ml) with or without growth factor (2 μg/ml human VEGF) is implanted subcutaneously on the dorsal flank of C57/C6 mice. The mice are treated with the test compound (e.g. 25, 50 or 100 mg/kg p.o. once daily) or vehicle starting on the day of implantation of the chamber and continuing for 4 days after. At the end of the treatment, the mice are killed, and the chambers are removed. The vascularized tissue growing around the chamber is carefully removed and weighed, and the blood content is assessed by measuring the hemoglobin content of the tissue (Drabkins method; Sigma, Deisenhofen, Germany). It has been shown previously that these growth factors induce dose-dependent increases in weight and blood content of this tissue growing (characterized histologically to contain fibroblasts and small blood vessels) around the chambers and that this response is blocked by antibodies that specifically neutralize VEGF (see Wood J M et al., Cancer Res. 60(8), 2178-2189, (2000); and Schlaeppi et al., J. Cancer Res. Clin. Oncol. 125, 336-342, (1999)).

In view of the high expression of the Tie-2 antagonist angiopoietin-2 expression of which is up-regulated at sites where angiogenesis takes place, this result is corroborating surprising former findings. In addition, although VEGF has been used to stimulate angiogenesis in the in vivo model, selective Tie-2 inhibitors are sufficient to inhibit angiogenesis. Therefore the compounds of the present invention can support treatments inhibiting VEGF-driven angiogenesis or replace them, especially where they are not successful, and thus are a very good addition to the arsenal of antitumor drugs and therapies.

Angiogenesis is regarded as a prerequisite for those tumors which grow beyond a maximum diameter of about 1-2 mm; up to this limit, oxygen and nutrients may be supplied to the tumor cells by diffusion. Every tumor, regardless of its origin and its cause, is thus dependent on angiogenesis for its growth after it has reached a certain size. Three principal mechanisms play an important role in the activity of angiogenesis inhibitors against tumors: 1) Inhibition of the growth of vessels, especially capillaries, into avascular resting tumors, with the result that there is no net tumor growth owing to the balance that is achieved between apoptosis and proliferation; 2) Prevention of the migration of tumor cells owing to the absence of blood flow to and from tumors; and 3) Inhibition of endothelial cell proliferation, thus avoiding the paracrine growth-stimulating effect exerted on the surrounding tissue by the endothelial cells normally lining the vessels.

In a preferred sense of the invention, a disease or disorder dependent on activity of a protein (preferably tyrosine) kinase, especially Tie-2, where a compound of the formula I can be used is one or more of a proliferative disease (meaning one dependent on inadequate including a hyperproliferative condition, such as one or more of leukemia, hyperplasia, fibrosis (especially pulmonary, but also other types of fibrosis, such as renal fibrosis or hepatic cirrhosis), angiogenesis, psoriasis, atherosclerosis and smooth muscle proliferation in the blood vessels, such as stenosis or restenosis following angioplasty. Further, a compound of the formula I may be used for the treatment of thrombosis and/or scleroderma.

Preferred is the use of a compound of the formula I in the therapy (including prophylaxis) of a proliferative disorder (especially which is dependent on (for example inadequate) Tie-2 activity) selected from tumor or cancer diseases, especially against preferably a benign or especially malignant tumor or cancer disease, more preferably solid tumors, e.g. carcinoma of the brain, kidney, liver, adrenal gland, bladder, breast, stomach (especially gastric tumors), ovaries, cervix, endometrium, colon, rectum, prostate, pancreas, lung (e.g. small or large cell lung carcinomas), vagina, thyroid, sarcoma, glioblastomas, myeloma, especially multiple myeloma or gastrointestinal cancer, especially colon carcinoma or colorectal adenoma, skin cancer, e.g. melanoma, Kaposi's sarcoma, a tumor of the neck and head, e.g. squameous carcinoma of the head and neck, including neoplasias, especially of epithelial character, e.g. in the case of mammary carcinoma; an epidermal hyperproliferation (other than cancer), especially psoriasis; prostate hyperplasia; malignant pleural mesotherioma; lymphoma; or further liquid tumors, e.g. leukemia.

A compound of formula I or its use makes it possible to bring about the regression of tumors and to prevent the formation of tumor metastases and the growth of (also micro)metastases.

Compounds of the formula I, in regard of their ability to inhibit Tie-2 kinase, and thus to modulate angiogenesis, are especially appropriate for the use against diseases or disorders related to the inadequate activity of Tie-2 kinase, especially an overexpression thereof.

The compounds of the formula I are especially of use to prevent or treat the mentioned and other diseases that are triggered by persistent angiogenesis, such as restenosis, e.g., stent-induced restenosis; Crohn's disease; Hodgkin's disease; malignant nephrosclerosis; thrombotic microangiopathic syndromes; (e.g. chronic) transplant rejections and glomerulopathy; mesangial cell-proliferative diseases; injuries of the nerve tissue; for inhibiting the re-occlusion of vessels after balloon catheter treatment, for use in vascular prosthetics or after inserting mechanical devices for holding vessels open, such as, e.g., stents, as immunosuppressants, as an aid in scar-free wound healing, and for treating age spots and contact dermatitis, diseases caused by ocular neovascularisation, especially (e.g. ischemic) retinopathies such as diabetic retinopathy, neovascular glaucoma or (e.g. age-related) macula degeneration, Von Hippel Lindau disease, hemangioblastoma, (haem)angioma, mesangial cell proliferative disorders such as chronic or acute renal diseases, e.g. diabetic nephropathy, obesity, malignant nephrosclerosis, thrombotic microangiopathy syndromes or trans-plant rejection, or especially inflammatory renal disease, such as glomerulonephritis, especially mesangioproliferative glomerulonephritis, haemolytic-uraemic syndrome, diabetic nephropathy, hypertensive nephrosclerosis, atheroma, arterial restenosis, autoimmune and/or inflammatory diseases, e.g. acute inflammation, rheumatoid arthritis, inflammatory bowel disease, rheumatoid inflammatory diseases or other chronic inflammatory disorders, diabetes, endometriosis, chronic asthma, arterial or post-transplantational atherosclerosis, neurodegenerative disorders, and especially neoplastic diseases such as cancers (especially solid tumours but also leukemias as mentioned above), myelodysplastic syndrome, AML (acute myeloid leukemia), AMM (agnogenic myeloid metaplasia), mesothelioma, glioma and glioblastoma.

Preferably, the invention relates to the use of compounds of the formula I, or pharmaceutically acceptable salts thereof, in the treatment of solid tumors as mentioned herein.

Where above or subsequently the term “use” is mentioned, this includes any one or more of the following embodiments of the invention, respectively: the use of a compound of the formula I in the treatment of (especially tyrosine) protein kinase dependent diseases, its use for the manufacture of pharmaceutical compositions for use in the treatment of said diseases, methods of use of a compound of the formula I in the treatment of said diseases, pharmaceutical preparations comprising a compound of the formula I for the treatment of said diseases, and a compound of the formula I for use in the treatment of said diseases, as appropriate and expedient, if not stated otherwise. In particular, diseases to be treated and are thus preferred for USE of a compound of formula (I) are selected from (especially tyrosine) protein kinase dependent (“dependent” meaning also “supported”, not only “solely dependent”) diseases mentioned above, especially corresponding proliferative diseases, more especially diseases that depend on Tie-2.

### Process of Manufacture

A compound of formula I can be prepared analogously to methods that, for other compounds, are in principle known in the art, so that for the novel compounds of the formula I the process is novel as analogy process, preferably by reacting

a) a compound of the formula II,

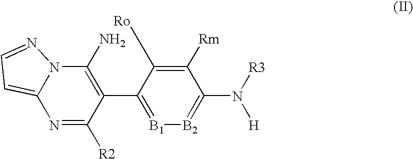

wherein R2, R3, B₁, B₂, Ro and Rm are as defined for a compound of the formula I, with an acid of the formula III,

R1-OH (III)

or a reactive derivative thereof, wherein R1 is as defined for a compound of the formula I, or
b) reacting a nitrile of the formula IV,

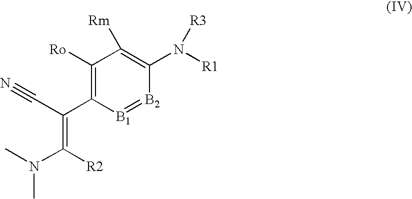

wherein R1, R2, R3, B₁, B₂, Ro and Rm are as defined for a compound of the formula I, with 3-aminopyrazole;
and, if desired, transforming a compound of formula I into a different compound of formula I, transforming a salt of an obtainable compound of formula I into the free compound or a different salt, transforming an obtainable free compound of formula I into a salt thereof, and/or separating an obtainable mixture of isomers of a compound of formula I into individual isomers.

The reaction under a) preferably can take place under customary conditions for the formation of amide bonds, and the acid of the formula III is either used as such and a reactive derivative is formed in situ, e.g. by dissolving the compounds of formulae II and III in a suitable solvent, for example N,N-dimethylformamide, N,N-dimethylacetamide, N-methyl-2-pyrrolidone, methylene chloride, tetrahydrofurane or a mixture of two or more such solvents, and/or at least one suitable base, for example triethylamine, diisopropylethylamine (DIEA), N-methylmorpholine or pyridine, together with a suitable coupling agent that forms a preferred reactive derivative of the carbonic acid of formula III in situ, for example dicyclohexylcarbodiimide/1-hydroxybenzotriazole (DCC/HOBT); O-(1,2-dihydro-2-oxo-1-pyridyl)-N,N,N′,N′-tetramethyluronium tetrafluoroborate (TPTU); O-benzotriazol-1-yl)-N,N,N′,N′-tetramethyluronium tetrafluoroborate (TBTU); or 1-(3-dimethylaminopropyl)-3-ethylcarbodiimide hydrochloride (EDC) (for a review of other possible coupling agents, see e.g. Klauser; Bodansky, Synthesis 1972, 453-463), preferably reacting at a temperature of between approximately −20 and 50° C., especially between 0° C. and room temperature, to yield a compound of formula I. Alternatively, the acid of the formula III is used in the form of a reactive derivative, e.g. as the acid halide, such as chloride, as an anhydride, as an active ester or amide, e.g. in the case of unsubstituted or substituted heterocyclylaminosulfonyl an 2-oxo-1,3-oxazolidino derivative or, if the moiety R1 to be introduced is a substituted aminocarbonyl moiety, especially unsubstituted or substituted C₆-C₁₄-arylaminoarbonyl or unsubstituted or substituted heterocyclylaminocarbonyl, using a corresponding isocyanate precursor where, during the reaction, the isocyanato group forms the aminocarbonyl group, preferably in the presence of a base and/or a solvent and at preferred temperatures as just described.

The reaction under b) preferably can take place in the presence of an organic acid, such as acetic acid, and an inorganic acid, such as hydrogenchloride, in an appropriate solvent, such as an alcohol, e.g. ethanol, at preferably elevated temperatures, e.g. from 50° C. to the reflux temperature of the reaction mixture.

### Optional Reactions and Conversions

Compounds of the formula I, or protected forms thereof directly obtained according to any one of the preceding procedures or after introducing protecting groups anew, which are included subsequently as starting materials for conversions as well even if not mentioned specifically, can be converted into different compounds of the formula I according to known procedures, where required followed removal of protecting groups.

In a compound of the formula I wherein R3 is hydrogen and the other symbols have the meanings defined under formula I, a moiety R3=unsubstituted or substituted alkyl can be introduced by reaction of a compound of the formula I wherein R3 is hydrogen with an alkylating agent, e.g. a compound of the formula V,

R3-G (V)

wherein R3 is unsubstituted or substituted alkyl and wherein G is a leaving group, such as halo, especially chloro, bromo or iodo, arylsulfonyloxy, such as toluolsulfonyloxy, or alkanesulfonyloxy, such as methansulfonyloxy, under customary reaction conditions and in the presence of appropriate solvents. If required, the 7-amino group at the central pyrazolo[1,5-a]pyrimidin ring can be protected before (also already on an intermediate stage) and deprotected after the alkylation in a customary way.

In the examples, appropriate reaction conditions can be found that may be used for analogous conversions of different compounds of the formula I.

Salts of compounds of formula I having at least one salt-forming group may be prepared in a manner known per se. For example, salts of compounds of formula I having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of formula I are obtained in customary manner, e.g. by treating the compounds with an acid or a suitable anion exchange reagent. Internal salts of compounds of formula I containing acid and basic salt-forming groups, e.g. a free carboxy group and a free amino group, may be formed, e.g. by the neutralisation of salts, such as acid addition salts, to the isoelectric point, e.g. with weak bases, or by treatment with ion exchangers.

A salt of a compound of the formula I can be converted in customary manner into the free compound; metal and ammonium salts can be converted, for example, by treatment with suitable acids, and acid addition salts, for example, by treatment with a suitable basic agent. In both cases, suitable ion exchangers may be used.

Stereoisomeric mixtures, e.g. mixtures of diastereomers, can be separated into their corresponding isomers in a manner known per se by means of appropriate separation methods. Diastereomeric mixtures for example may be separated into their individual diastereomers by means of fractionated crystallization, chromatography, solvent distribution, and similar procedures. This separation may take place either at the level of one of the starting compounds or in a compound of formula I itself. Enantiomers may be separated through the formation of diastereomeric salts, for example by salt formation with an enantiomer-pure chiral acid, or by means of chromatography, for example by HPLC, using chromatographic substrates with chiral ligands.

Intermediates and final products can be worked up and/or purified according to standard methods, e.g. using chromatographic methods, distribution methods, (re-) crystallization, and the like.

### Starting Materials

Starting Materials, including intermediates, for compounds of the formula I, such as the compounds of the formulae II, III and IV, can be prepared, for example, according to methods that are known in the art, according to methods described in the examples and the section below with the title Examples, or to methods analogous to those described in the examples or the section below with the title Examples, and/or they are known or commercially available.

In the subsequent description of starting materials and intermediates and their synthesis, R1, R2, R3, B₁, B₂, Ro and Rm have the meanings given above for the corresponding starting materials or herein for compounds of the formula I or especially in the Examples for the respective starting materials or intermediates, if not indicated otherwise directly or by the context. Protecting groups, if not specifically mentioned, can be introduced and removed at appropriate steps in order to prevent functional groups, the reaction of which is not desired in the corresponding reaction step or steps, employing protecting groups, methods for their introduction and their removal are as described above or below, e.g. in the references mentioned under “General Process Conditions”. The person skilled in the art will readily be able to decide whether and which protecting groups are useful or required.

A compound of the formula II can, for example, be prepared as or in analogy to the method described in General scheme-2 in the “Examples” Section for INT5 which falls under formula II wherein R3 is hydrogen. A corresponding compound of the formula II wherein R3 is unsubstituted or substituted lower alkyl can, for example, be prepared using a compound of the formula V under reaction conditions analogous to those described under “Optional reactions and conversions”.

A compound of the formula IV can, for example, be prepared as or in analogy to the method shown in General scheme-1 in the “Examples” Section for the compounds INT3-1, INT3-2, INT3-3 and INT3-4 which all fall under formula IV. A corresponding compound of the formula IV wherein R3 is unsubstituted or substituted lower alkyl can, for example, be prepared using a compound of the formula V under reaction conditions analogous to those described under “Optional reactions and conversions”.

Other starting materials, e.g. those of the formula III or V, are known in the art, commercially available and/or can be prepared according to standard procedures, e.g. in analogy to or by methods described in the Examples.

### General Process Conditions

The following applies in general to all processes mentioned hereinbefore and hereinafter, while reaction conditions specifically mentioned above or below are preferred:

In any of the reactions mentioned hereinbefore and hereinafter, protecting groups may be used where appropriate or desired, even if this is not mentioned specifically, to protect functional groups that are not intended to take part in a given reaction, and they can be introduced and/or removed at appropriate or desired stages. Reactions comprising the use of protecting groups are therefore included as possible wherever reactions without specific mentioning of protection and/or deprotection are described in this specification.

Within the scope of this disclosure only a readily removable group that is not a constituent of the particular desired end product of formula I is designated a “protecting group”, unless the context indicates otherwise. The protection of functional groups by such protecting groups, the protecting groups themselves, and the reactions appropriate for their removal are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage).

All the above-mentioned process steps can be carried out under reaction conditions that are known per se, preferably those mentioned specifically, in the absence or, customarily, in the presence of solvents or diluents, preferably solvents or diluents that are inert towards the reagents used and dissolve them, in the absence or presence of catalysts, condensation or neutralizing agents, for example ion exchangers, such as cation exchangers, e.g. in the H⁺ form, depending on the nature of the reaction and/or of the reactants at reduced, normal or elevated temperature, for example in a temperature range of from about −100° C. to about 190° C., preferably from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofurane or dioxane, liquid aromatic hydrocarbons, such as benzene or toluene, alcohols, such as methanol, ethanol or 1- or 2-propanol, nitriles, such as acetonitrile, halogenated hydrocarbons, e.g. as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of these, for example aqueous solutions, unless otherwise indicated in the description of the processes. Such solvent mixtures may also be used in working up, for example by chromatography or partitioning.

Intermediates and final products can be worked up and/or purified according to standard methods, e.g. using chromatographic methods, distribution methods, (re-) crystallization, distillation (under normal or reduced pressure), steam distillation and the like.

The invention relates also to those forms of the process in which a compound obtainable as intermediate at any stage of the process is used as starting material and the remaining process steps are carried out, or in which a starting material is formed under the reaction conditions or is used in the form of a derivative, for example in protected form or in the form of a salt, or a compound obtainable by the process according to the invention is produced under the process conditions and processed further in situ. In the process of the present invention those starting materials are preferably used which result in compounds of formula I described as being preferred. Special preference is given to reaction conditions that are identical or analogous to those mentioned in the Examples. The invention also relates to novel starting materials.

### PREFERRED EMBODIMENTS ACCORDING TO THE INVENTION

In the following preferred embodiments as well as in preceding and following embodiments of more general scope, any one or more or all general expressions can be replaced by the corresponding more specific definitions provided above and below, thus yielding stronger preferred embodiments of the invention.

The invention, in one preferred embodiment, relates to a compound of the formula I wherein

R1 is unsubstituted or substituted heterocyclylaminocarbonyl wherein heterocyclyl has 3 to 14 ring atoms, unsubstituted or substituted C₆-C₁₄-arylaminosulfonyl, unsubstituted or substituted heterocyclylaminosulfonyl wherein heterocyclyl has 3 to 14 ring atoms, unsubstituted or substituted lower-alkanesulfonyl, unsubstituted or substituted C₆-C₁₄-arylsulfonyl, unsubstituted or substituted heterocyclylsulfonyl wherein heterocyclyl has 3 to 14 ring atoms, or unsubstituted or substituted C₆-C₁₄-arylcarbonyl;
R2 is hydrogen, lower alkyl, heterocyclyl-lower alkyl wherein heterocyclyl is unsubstituted or substituted and has 3 to 14 ring atoms, hydroxyl-lower alkyl, esterified or etherified hydroxyl-lower alkyl or unsubstituted or substituted amino-lower alkyl;
R3 is hydrogen or unsubstituted or substituted lower alkyl,

### B₁ is N or CRo;

### B₂ is N or CRm;

and each Ro and Rm, independently of the others, is selected from hydrogen, lower alkyl, halo and lower alkoxy;
or a (preferably pharmaceutically acceptable) salt thereof.

The invention, in another specific embodiment, relates to a compound of the formula I wherein R1 is substituted C₆-C₁₄-arylaminocarbonyl wherein the substituents are selected from C₁-C₇-alkyl, hydroxy-C₁-C₇-alkyl, C₁-C₇-alkoxy-C₁-C₇-alkyl, amino-C₁-C₇-alkyl, N-mono- or N,N-di-(C₁-C₇-alkyl and/or mono-C₁-C₇-alkoxy-C₁-C₇alkyl and/or (mono- or di-(C₁-C₇-alkyl)amino-C₁-C₇-alkyl)-amino-C₁-C₇-alkyl, lower alkoxy, cyano and preferably halo, especially fluoro, chloro (which is most preferred) or bromo, hydroxy, C₁-C₇-alkoxy, phenyl-C₁-C₇-alkoxy wherein phenyl is unsubstituted or substituted by C₁-C₇-alkoxy and/or halo; or a (preferably pharmaceutically acceptable) salt thereof.

Another preferred embodiment of the invention relates to a compound of the formula I wherein

R1 is unsubstituted or substituted heterocyclylaminocarbonyl wherein heterocyclyl has 3 to 14 ring atoms;
R2 is hydrogen, lower alkyl, heterocyclyl-lower alkyl wherein heterocyclyl is unsubstituted or substituted and has 3 to 14 ring atoms, hydroxyl-lower alkyl, esterified or etherified hydroxyl-lower alkyl or unsubstituted or substituted amino-lower alkyl;
R3 is hydrogen or unsubstituted or substituted lower alkyl;

### B₁ is N or CRo;

### B₂ is N or CRm;

and each Ro and Rm, independently of the others, is selected from hydrogen, lower alkyl, halo and lower alkoxy;
or a salt thereof.

A yet more preferred embodiment of the invention relates to a compound of the formula I, wherein

R1 is phenylaminocarbonyl wherein phenyl is unsubstituted or substituted by one or more moieties independently selected from lower alkyl, halo (very preferred), especially chloro; lower alkoxy and cyano;
pyrazolyl-aminocarbonyl or isoxazolylaminocarbonyl where pyrazolyl or isoxazolyl is unsubstituted or substituted by one or two moieties independently selected from the group consisting of lower alkyl and phenyl that is unsubstituted or substituted with halo, lower alkoxy, piperazino-lower alkyl, 4-lower alkylpiperazino-lower alkyl and morpholino-lower alkyl; pyrazolyl-aminosulfonyl or isoxazolylaminosulfonyl, where each pyrazolyl or isoxazolyl is unsubstituted or substituted by one or two moieties independently selected from the group consisting of lower alkyl and phenyl that is unsubstituted or substituted with halo, lower alkoxy, piperazino-lower alkyl, 4-lower alkylpiperazino-lower alkyl and morpholino-lower alkyl; phenyl-lower alkanesulfonyl, wherein phenyl is unsubstituted (preferred) or substituted with one or more, e.g. up to three, moieties independently selected from the group consisting of lower alkyl, halo (especially preferred), halo-lower alkyl, lower alkoxy and cyano; phenylsulfonyl wherein the phenyl is unsubstituted or substituted by one or more moieties independently selected from the group consisting of lower alkyl, halo (preferred), halo-lower alkyl, lower alkoxy and cyano;
R2 is hydrogen, lower alkyl, especially methyl, piperazino-lower alkyl, especially piperazinomethyl, 4-lower alkyl-piperazino-lower alkyl, especially 4-methyl-piperazino-methyl, lower alkoxy-lower alkyl, especially lower-alkoxymethyl or phenyl-lower alkoxy-lower alkyl, especially benzyloxymethyl;
R3 is hydrogen (preferred) or lower alkyl,

### B₁ is N or CRo;

### B₂ is CRm;

and each Ro and Rm, independently of the others, is selected from hydrogen, lower alkyl, especially methyl, halo, especially fluoro or chloro, and lower alkoxy, especially methoxy;
or a (preferably pharmaceutically acceptable) salt thereof.

Most preferred is a compound of the formula I, or a (preferably pharmaceutically acceptable) salt thereof, as exemplified herein-below under ‘Examples’, or its USE as defined above.

### Pharmaceutical Compositions

The invention relates also to pharmaceutical compositions comprising a (preferably novel) compound of formula I, to their use in the therapeutic (in a broader aspect of the invention also prophylactic) treatment or a method of treatment of a disease or disorder that depends on inadequate protein (especially Tie-2) kinase activity, especially the preferred disorders or diseases mentioned above, to the compounds for said use and to pharmaceutical preparations and their manufacture, especially for said uses. More generally, pharmaceutical preparations are useful in case of compounds of the formula I.

The pharmacologically acceptable compounds of the present invention may be present in or employed, for example, for the preparation of pharmaceutical compositions that comprise an effective amount of a compound of the formula I, or a pharmaceutically acceptable salt thereof, as active ingredient together or in admixture with one or more inorganic or organic, solid or liquid, pharmaceutically acceptable carriers (carrier materials).

The invention relates also to a pharmaceutical composition that is suitable for administration to a warm-blooded animal, especially a human (or to cells or cell lines derived from a warm-blooded animal, especially a human, e.g. lymphocytes), for the treatment (this, in a broader aspect of the invention, also including prevention of (=prophylaxis against)) a disease that responds to inhibition of protein (especially Tie-2) kinase activity, comprising an amount of a compound of formula I or a pharmaceutically acceptable salt thereof, preferably which is effective for said inhibition, together with at least one pharmaceutically acceptable carrier.

The pharmaceutical compositions according to the invention are those for enteral, such as nasal, rectal or oral, or parenteral, such as intramuscular or intravenous, administration to warm-blooded animals (especially a human), that comprise an effective dose of the pharmacologically active ingredient, alone or together with a significant amount of a pharmaceutically acceptable carrier. The dose of the active ingredient depends on the species of warm-blooded animal, the body weight, the age and the individual condition, individual pharmacokinetic data, the disease to be treated and the mode of administration.

The invention relates also to method of treatment for a disease that responds to inhibition of a disease that depends on inadequate activity of a protein (especially Tie-2) kinase; which comprises administering a prophylactically or especially therapeutically effective amount of a compound of formula I, or a pharmaceutically acceptable salt thereof, especially to a warm-blooded animal, for example a human, that, on account of one of the mentioned diseases, requires such treatment.

The dose of a compound of the formula I or a pharmaceutically acceptable salt thereof to be administered to warm-blooded animals, for example humans of approximately 70 kg body weight, preferably is from approximately 3 mg to approximately 10 g, more preferably from approximately 10 mg to approximately 1.5 g, most preferably from about 100 mg to about 1000 mg/person/day, divided preferably into 1-3 single doses which may, for example, be of the same size. Usually, children receive half of the adult dose.

The pharmaceutical compositions comprise from approximately 1% to approximately 95%, preferably from approximately 20% to approximately 90%, active ingredient. Pharmaceutical compositions according to the invention may be, for example, in unit dosage form, such as in the form of ampoules, vials, suppositories, dragées, tablets or capsules.

The pharmaceutical compositions of the present invention are prepared in a manner known per se, for example by means of conventional dissolving, lyophilizing, mixing, granulating or confectioning processes.

Solutions of the active ingredient, and also suspensions, and especially isotonic aqueous solutions or suspensions, are preferably used, it being possible, for example in the case of lyophilized compositions that comprise the active ingredient alone or together with a carrier, for example mannitol, for such solutions or suspensions to be produced prior to use. The pharmaceutical compositions may be sterilized and/or may comprise excipients, for example preservatives, stabilizers, wetting and/or emulsifying agents, solubilizers, salts for regulating the osmotic pressure and/or buffers, and are prepared in a manner known per se, for example by means of conventional dissolving or lyophilizing processes. The said solutions or suspensions may comprise viscosity-increasing substances, such as sodium carboxymethylcellulose, carboxymethylcellulose, dextran, polyvinylpyrrolidone or gelatin.

Suspensions in oil comprise as the oil component the vegetable, synthetic or semi-synthetic oils customary for injection purposes. There may be mentioned as such especially liquid fatty acid esters that contain as the acid component a long-chained fatty acid having from 8-22, especially from 12-22, carbon atoms, for example lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brasidic acid or linoleic acid, if desired with the addition of antioxidants, for example vitamin E,β-carotene or 3,5-di-tert-butyl-4-hydroxytoluene. The alcohol component of those fatty acid esters has a maximum of 6 carbon atoms and is a mono- or poly-hydroxy, for example a mono-, di- or tri-hydroxy, alcohol, for example methanol, ethanol, propanol, butanol or pentanol or the isomers thereof, but especially glycol and glycerol. The following examples of fatty acid esters are therefore to be mentioned: ethyl oleate, isopropyl myristate, isopropyl palmitate, “Labrafil M 2375” (polyoxyethylene glycerol trioleate, Gattefossé, Paris), “Miglyol 812” (triglyceride of saturated fatty acids with a chain length of C8 to C12, Hüls AG, Germany), but especially vegetable oils, such as cottonseed oil, almond oil, olive oil, castor oil, sesame oil, soybean oil and groundnut oil.

The injection or infusion compositions are prepared in customary manner under sterile conditions; the same applies also to introducing the compositions into ampoules or vials and sealing the containers.

Pharmaceutical compositions for oral administration can be obtained by combining the active ingredient with solid carriers, if desired granulating a resulting mixture, and processing the mixture, if desired or necessary, after the addition of appropriate excipients, into tablets, dragée cores or capsules. It is also possible for them to be incorporated into plastics carriers that allow the active ingredients to diffuse or be released in measured amounts.

Suitable carriers are especially fillers, such as sugars, for example lactose, saccharose, mannitol or sorbitol, cellulose preparations and/or calcium phosphates, for example tricalcium phosphate or calcium hydrogen phosphate, and binders, such as starch pastes using for example corn, wheat, rice or potato starch, gelatin, tragacanth, methylcellulose, hydroxypropylmethylcellulose, sodium carboxymethylcellulose and/or polyvinylpyrrolidone, and/or, if desired, disintegrators, such as the above-mentioned starches, and/or carboxymethyl starch, crosslinked polyvinylpyrrolidone, agar, alginic acid or a salt thereof, such as sodium alginate. Excipients are especially flow conditioners and lubricants, for example silicic acid, talc, stearic acid or salts thereof, such as magnesium or calcium stearate, and/or polyethylene glycol. Dragée cores are provided with suitable, optionally enteric, coatings, there being used, inter alia, concentrated sugar solutions which may comprise gum arabic, talc, polyvinylpyrrolidone, polyethylene glycol and/or titanium dioxide, or coating solutions in suitable organic solvents, or, for the preparation of enteric coatings, solutions of suitable cellulose preparations, such as ethylcellulose phthalate or hydroxypropylmethylcellulose phthalate. Capsules are dry-filled capsules made of gelatin and soft sealed capsules made of gelatin and a plasticizer, such as glycerol or sorbitol. The dry-filled capsules may comprise the active ingredient in the form of granules, for example with fillers, such as lactose, binders, such as starches, and/or glidants, such as talc or magnesium stearate, and if desired with stabilizers. In soft capsules the active ingredient is preferably dissolved or suspended in suitable oily excipients, such as fatty oils, paraffin oil or liquid polyethylene glycols, it being possible also for stabilizers and/or antibacterial agents to be added. Dyes or pigments may be added to the tablets or dragée coatings or the capsule casings, for example for identification purposes or to indicate different doses of active ingredient.

A compound of the formula I may also be used to advantage in combination with other antiproliferative agents. Such antiproliferative agents include, but are not limited to aromatase inhibitors; antiestrogens; topoisomerase I inhibitors; topoisomerase II inhibitors; microtubule active agents; alkylating agents; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; bisphosphonates; biological response modifiers; antiproliferative antibodies; heparanase inhibitors; inhibitors of Ras oncogenic isoforms; telomerase inhibitors; proteasome inhibitors; agents used in the treatment of hematologic malignancies; compounds which target, decrease or inhibit the activity of Flt-3; Hsp90 inhibitors; and temozolomide (TEMODAL®).

The term “aromatase inhibitor” as used herein relates to a compound which inhibits the estrogen production, i.e. the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane can be administered, e.g., in the form as it is marketed, e.g. under the trademark AROMASIN. Formestane can be administered, e.g., in the form as it is marketed, e.g. under the trademark LENTARON. Fadrozole can be administered, e.g., in the form as it is marketed, e.g. under the trademark AFEMA. Anastrozole can be administered, e.g., in the form as it is marketed, e.g. under the trademark ARIMIDEX. Letrozole can be administered, e.g., in the form as it is marketed, e.g. under the trademark FEMARA or FEMAR. Aminoglutethimide can be administered, e.g., in the form as it is marketed, e.g. under the trademark ORIMETEN. A combination of the invention comprising a chemotherapeutic agent which is an aromatase inhibitor is particularly useful for the treatment of hormone receptor positive tumors, e.g. breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g. under the trademark NOLVADEX. Raloxifene hydrochloride can be administered, e.g., in the form as it is marketed, e.g. under the trademark EVISTA. Fulvestrant can be formulated as disclosed in U.S. Pat. No. 4,659,516 or it can be administered, e.g., in the form as it is marketed, e.g. under the trademark FASLODEX. A combination of the invention comprising a chemotherapeutic agent which is an antiestrogen is particularly useful for the treatment of estrogen receptor positive tumors, e.g. breast tumors.

The term “anti-androgen” as used herein relates to any substance which is capable of inhibiting the biological effects of androgenic hormones and includes, but is not limited to, bicalutamide (CASODEX), which can be formulated, e.g. as disclosed in U.S. Pat. No. 4,636,505.

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX. Abarelix can be formulated, e.g. as disclosed in U.S. Pat. No. 5,843,901.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148 (compound A1 in WO99/17804). Irinotecan can be administered, e.g. in the form as it is marketed, e.g. under the trademark CAMPTOSAR. Topotecan can be administered, e.g., in the form as it is marketed, e.g. under the trademark HYCAMTIN.

The term “topoisomerase II inhibitor” as used herein includes, but is not limited to the anthracyclines such as doxorubicin (including liposomal formulation, e.g. CAELYX), daunorubicin, epirubicin, idarubicin and nemorubicin, the anthraquinones mitoxantrone and losoxantrone, and the podophillotoxines etoposide and teniposide. Etoposide can be administered, e.g. in the form as it is marketed, e.g. under the trademark ETOPOPHOS. Teniposide can be administered, e.g. in the form as it is marketed, e.g. under the trademark VM 26-BRISTOL. Doxorubicin can be administered, e.g. in the form as it is marketed, e.g. under the trademark ADRIBLASTIN or ADRIAMYCIN. Epirubicin can be administered, e.g. in the form as it is marketed, e.g. under the trademark FARMORUBICIN. Idarubicin can be administered, e.g. in the form as it is marketed, e.g. under the trademark ZAVEDOS. Mitoxantrone can be administered, e.g. in the form as it is marketed, e.g. under the trademark NOVANTRON.

The term “microtubule active agent” relates to microtubule stabilizing, microtubule destabilizing agents and microtublin polymerization inhibitors including, but not limited to taxanes, e.g. paclitaxel and docetaxel, vinca alkaloids, e.g., vinblastine, especially vinblastine sulfate, vincristine especially vincristine sulfate, and vinorelbine, discodermolides, cochicine and epothilones and derivatives thereof, e.g. epothilone B or a derivative thereof. Paclitaxel may be administered e.g. in the form as it is marketed, e.g. TAXOL. Docetaxel can be administered, e.g., in the form as it is marketed, e.g. under the trademark TAXOTERE. Vinblastine sulfate can be administered, e.g., in the form as it is marketed, e.g. under the trademark VINBLASTIN R.P. Vincristine sulfate can be administered, e.g., in the form as it is marketed, e.g. under the trademark FARMISTIN. Discodermolide can be obtained, e.g., as disclosed in U.S. Pat. No. 5,010,099. Also included are Epothilone derivatives which are disclosed in WO 98/10121, U.S. Pat. No. 6,194,181, WO 98/25929, WO 98/08849, WO 99/43653, WO 98/22461 and WO 00/31247. Especially preferred are Epothilone A and/or B.

The term “alkylating agent” as used herein includes, but is not limited to, cyclophosphamide, ifosfamide, melphalan or nitrosourea (BCNU or Gliadel). Cyclophosphamide can be administered, e.g., in the form as it is marketed, e.g. under the trademark CYCLOSTIN. Ifosfamide can be administered, e.g., in the form as it is marketed, e.g. under the trademark HOLOXAN.

The term “histone deacetylase inhibitors” or “HDAC inhibitors” relates to compounds which inhibit the histone deacetylase and which possess antiproliferative activity. This includes compounds disclosed in WO 02/22577, especially N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(2-methyl-1H-indol-3-yl)-ethyl]-amino]methyl]phenyl]-2E-2-propenamide and pharmaceutically acceptable salts thereof. It further especially includes Suberoylanilide hydroxamic acid (SAHA).

The term “antineoplastic antimetabolite” includes, but is not limited to, 5-Fluorouracil or 5-FU, capecitabine, gemcitabine, DNA demethylating agents, such as 5-azacytidine and decitabine, methotrexate and edatrexate. Capecitabine can be administered, e.g., in the form as it is marketed, e.g. under the trademark XELODA. Gemcitabine can be administered, e.g., in the form as it is marketed, e.g. under the trademark GEMZAR. Also included is the monoclonal antibody trastuzumab which can be administered, e.g., in the form as it is marketed, e.g. under the trademark HERCEPTIN.

The term “platin compound” as used herein includes, but is not limited to, carboplatin, cisplatin, cisplatinum and oxaliplatin. Carboplatin can be administered, e.g., in the form as it is marketed, e.g. under the trademark CARBOPLAT. Oxaliplatin can be administered, e.g., in the form as it is marketed, e.g. under the trademark ELOXATIN.

The term “compounds targeting/decreasing a protein or lipid kinase activity and further anti-angiogenic compounds” as used herein includes, but is not limited to: protein tyrosine kinase and/or serine and/or threonine kinase inhibitors or lipid kinase inhibitors, e.g.:

a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, e.g. a N-phenyl-2-pyrimidine-amine derivative, e.g. imatinib, SU101, SU6668, and GFB-111;
b) compounds targeting, decreasing or inhibiting the activity of the fibroblast growth factor-receptors (FGFR);
c) compounds targeting, decreasing or inhibiting the activity of the insulin-like growth factor receptor 1 (IGF-1R), such as compounds which target, decrease or inhibit the activity of IGF-IR, especially compounds which inhibit the IGF-1R receptor, such as those compounds disclosed in WO 02/092599;
d) compounds targeting, decreasing or inhibiting the activity of the Trk receptor tyrosine kinase family;
e) compounds targeting, decreasing or inhibiting the activity of the Axl receptor tyrosine kinase family;
f) compounds targeting, decreasing or inhibiting the activity of the c-Met receptor;
g) compounds targeting, decreasing or inhibiting the activity of the c-Kit receptor tyrosine kinases—(part of the PDGFR family), such as compounds which target, decrease or inhibit the activity of the c-Kit receptor tyrosine kinase family, especially compounds which inhibit the c-Kit receptor, e.g. imatinib;
h) compounds targeting, decreasing or inhibiting the activity of members of the c-Abl family and their gene-fusion products (e.g. BCR-Abl kinase), such as compounds which target, decrease or inhibit the activity of c-Abl family members and their gene fusion products, e.g. a N-phenyl-2-pyrimidine-amine derivative, e.g. imatinib; PD180970; AG957; NSC 680410; or PD173955 from ParkeDavis;
i) compounds targeting, decreasing or inhibiting the activity of members of the protein kinase C (PKC) and Raf family of serine/threonine kinases, members of the MEK, SRC, JAK, FAK, PDK and Ras/MAPK family members, or PI(3) kinase family, or of the PI(3)-kinase-related kinase family, and/or members of the cyclin-dependent kinase family (CDK) and are especially those staurosporine derivatives disclosed in U.S. Pat. No. 5,093,330, e.g. midostaurin; examples of further compounds include e.g. UCN-01, safingol, BAY 43-9006, Bryostatin 1, Perifosine; Ilmofosine; RO 318220 and RO 320432; GO 6976; Isis 3521; LY333531/LY379196; isochinoline compounds such as those disclosed in WO 00/09495; FTIs; PD184352 or QAN697 (a PI3K inhibitor);
j) compounds targeting, decreasing or inhibiting the activity of a protein-tyrosine kinase, such as imatinib mesylate (GLIVEC/GLEEVEC) or tyrphostin. A tyrphostin is preferably a low molecular weight (Mr<1500) compound, or a pharmaceutically acceptable salt thereof, especially a compound selected from the benzylidenemalonitrile class or the S-arylbenzenemalonirile or bisubstrate quinoline class of compounds, more especially any compound selected from the group consisting of Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin);
k) compounds targeting, decreasing or inhibiting the activity of the epidermal growth factor family of receptor tyrosine kinases (EGFR, ErbB2, ErbB3, ErbB4 as homo- or heterodimers), such as compounds which target, decrease or inhibit the activity of the epidermal growth factor receptor family are especially compounds, proteins or antibodies which inhibit members of the EGF receptor tyrosine kinase family, e.g. EGF receptor, ErbB2, ErbB3 and ErbB4 or bind to EGF or EGF related ligands, and are in particular those compounds, proteins or monoclonal antibodies generically and specifically disclosed in WO 97/02266, e.g. the compound of ex. 39, or in EP 0 564 409, WO 99/03854, EP 0520722, EP 0 566 226, EP 0 787 722, EP 0 837 063, U.S. Pat. No. 5,747,498, WO 98/10767, WO 97/30034, WO 97/49688, WO 97/38983 and, especially, WO 96/30347 (e.g. compound known as CP 358774), WO 96/33980 (e.g. compound ZD 1839) and WO 95/03283 (e.g. compound ZM105180); e.g. trastuzumab (HerpetinR), cetuximab, Iressa, OSI-774, CI-1033, EKB-569, GW-2016, E1.1, E2.4, E2.5, E6.2, E6.4, E2.11, E6.3 or E7.6.3, and 7H-pyrrolo-[2,3-d]pyrimidine derivatives which are disclosed in WO 03/013541; and
l) compounds targeting, decreasing or inhibiting the activity of the vascular endothelial growth factor-receptors (VEGFR), such as PTK-787 or Avastin.

Further anti-angiogenic compounds include compounds having another mechanism for their activity, e.g. unrelated to protein or lipid kinase inhibition e.g. thalidomide (THALOMID) and TNP-470 or RAD001.

Compounds which target, decrease or inhibit the activity of a protein or lipid phosphatase are e.g. inhibitors of phosphatase 1, phosphatase 2A, PTEN or CDC25, e.g. okadaic acid or a derivative thereof.

Compounds which induce cell differentiation processes are e.g. retinoic acid, α- γ- or δ-tocopherol or α- γ- or δ-tocotrienol.

The term “cyclooxygenase inhibitor” as used herein includes, but is not limited to, e.g. Cox-2 inhibitors, 5-alkyl substituted 2-arylaminophenylacetic acid and derivatives, such as celecoxib (CELEBREX), rofecoxib (VIOXX), etoricoxib, valdecoxib or a 5-alkyl-2-arylaminophenylacetic acid, e.g. 5-methyl-2-(2′-chloro-6′-fluoroanilino)phenyl acetic acid, lumiracoxib.

The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “bisphosphonates” as used herein includes, but is not limited to, etridonic, clodronic, tiludronic, pamidronic, alendronic, ibandronic, risedronic and zoledronic acid. “Etridonic acid” can be administered, e.g., in the form as it is marketed, e.g. under the trademark DIDRONEL. “Clodronic acid” can be administered, e.g., in the form as it is marketed, e.g. under the trademark BONEFOS. “Tiludronic acid” can be administered, e.g., in the form as it is marketed, e.g. under the trademark SKELID. “Pamidronic acid” can be administered, e.g. in the form as it is marketed, e.g. under the trademark AREDIA™. “Alendronic acid” can be administered, e.g., in the form as it is marketed, e.g. under the trademark FOSAMAX. “Ibandronic acid” can be administered, e.g., in the form as it is marketed, e.g. under the trademark BONDRANAT. “Risedronic acid” can be administered, e.g., in the form as it is marketed, e.g. under the trademark ACTONEL. “Zoledronic acid” can be administered, e.g. in the form as it is marketed, e.g. under the trademark ZOMETA.

The term “heparanase inhibitor” as used herein refers to compounds which target, decrease or inhibit heparin sulphate degradation. The term includes, but is not limited to, PI-88.

The term “biological response modifier” as used herein refers to a lymphokine or interferons, e.g. interferon γ.

The term “inhibitor of Ras oncogenic isoforms”, e.g. H-Ras, K-Ras, or N-Ras, as used herein refers to compounds which target, decrease or inhibit the oncogenic activity of Ras e.g. a “farnesyl transferase inhibitor”, e.g. L-744832, DK8G557 or R115777 (Zarnestra).

The term “telomerase inhibitor” as used herein refers to compounds which target, decrease or inhibit the activity of telomerase. Compounds which target, decrease or inhibit the activity of telomerase are especially compounds which inhibit the telomerase receptor, e.g. telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase are e.g. bengamide or a derivative thereof.

The term “proteasome inhibitor” as used herein refers to compounds which target, decrease or inhibit the activity of the proteasome. Compounds which target, decrease or inhibit the activity of the proteasome include e.g. PS-341 and MLN 341.

The term “matrix metalloproteinase inhibitor” or (“MMP inhibitor”) as used herein includes, but is not limited to collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or MJ996.

The term “agents used in the treatment of hematologic malignancies” as used herein includes, but is not limited to FMS-like tyrosine kinase inhibitors e.g. compounds targeting, decreasing or inhibiting the activity of Flt-3; interferon, 1-b-D-arabinofuransylcytosine (ara-c) and bisulfan; and ALK inhibitors e.g. compounds which target, decrease or inhibit anaplastic lymphoma kinase.

The term “compounds which target, decrease or inhibit the activity of Flt-3” are especially compounds, proteins or antibodies which inhibit Flt-3, e.g. PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteasome pathway.

Compounds targeting, decreasing or inhibiting the intrinsic ATPase activity of HSP90 are especially compounds, proteins or antibodies which inhibit the ATPase activity of HSP90 e.g., 17-allylamino, 17-demethoxygeldanamycin (17MG), a geldanamycin derivative; other geldanamycin related compounds; radicicol and HDAC inhibitors.

The term “antiproliferative antibodies” as used herein includes, but is not limited to trastuzumab (Herceptin™), Trastuzumab-DM1, erlotinib (Tarceva™), bevacizumab (Avastin™), rituximab (Rituxan®), PRO64553 (anti-CD40) and 2C4 Antibody. By antibodies is meant e.g. intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of formula I can be used in combination with standard leukemia therapies, especially in combination with therapies used for the treatment of AML. In particular, compounds of formula I can be administered in combination with e.g. farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

The structure of the active agents identified by code nos., generic or trade names may be taken from the actual edition of the standard compendium “The Merck Index” or from databases, e.g. Patents International (e.g. IMS World Publications).

The above-mentioned compounds, which can be used in combination with a compound of the formula I, can be prepared and administered as described in the art such as in the documents cited above.

A compound of the formula I may also be used to advantage in combination with known therapeutic processes, e.g., the administration of hormones or especially radiation.

A compound of formula I may in particular be used as a radiosensitizer, especially for the treatment of tumors which exhibit poor sensitivity to radiotherapy.

By “combination”, there is meant either a fixed combination in one dosage unit form, or a kit of parts for the combined administration where a compound of the formula I and a combination partner may be administered independently at the same time or separately within time intervals that especially allow that the combination partners show a cooperative, e.g. synergistic, effect, or by making use of administration schedules representing any combination thereof.

### EXAMPLES

The following examples serve to illustrate the invention without limiting the scope thereof:

### Abbreviations

Ac acetyl
aq. aqueous
Boc tert-butoxycarbonyl
Brine saturated sodium chloride solution
Celite trademark of Celite Corp. for filtering aid based on kieselguhr
conc. concentrated
DCM dichloromethane
DEAD diethyl azodicarboxylate
DMF N,N-dimethylformamide
DMSO dimethylsulfoxide
DMT-MM 4-(4,6-dimethoxy-1,3,5-triazin-2-yl)-4-methylmorpholinium chloride
EDC 1-(3-Dimethylaminopropyl)-3-ethylcarbodiimide hydrochloride
ES-MS electrospray mass spectrometry
Et ethyl
EtOAc ethyl acetate
h hour(s)
HOAt 1-hydroxy-7-azabenzotriazole
HPLC high-pressure liquid chromatography
HyFlo diatomaceous earth based filtering aid
IPr isopropyl
LAH lithium aluminium hydride
Me methyl
min minute(s)
mL milliliter(s)
MS Mass Spectrometry
NaOMe sodium methoxylate
NMR nuclear magnetic resonance
Ph phenyl
RT room temperature
TBTU O-(benzotriazol-1-yl)-N,N,N′,N′-tetramethylammonium tetrafluoroborate
TFA trifluoroacetic acid
THF tetrahydrofurane
TMS trimethylsilyl
WSCD=EDC

### Synthesis

Flash chromatography is performed by using silica gel (Merck; 40-63 μm). For thin layer chromatography, pre-coated silica gel (Merck 60 F254; Merck KgaA, Darmstadt, Germany)) plates are used. ¹NMR measurements are performed on a Varian Gemini 400 or Varian Gemini 300 spectrometer using tetramethylsilane as internal standard. Chemical shifts (δ) are expressed in ppm downfield from tetramethylsilane. Electrospray mass spectra are obtained with a Fisons Instruments VG Platform II. Commercially available solvents and chemicals are used for syntheses.

### HPLC Condition A:

### Column: Nucleosil 100-3 C18, 70×4.0 mm.

Flow rate: 1.0 ml/min
Mobile phase: A) TFA/water (0.1/100, v/v), B) TFA/acetonitrile (0.1/100, v/v)
Gradient: linear gradient from 20% B to 100% B in 7 min

### Detection: UV at 215 nm

### HPLC Condition B:

### Column: Speed ROD RP18e, 50×4.6 mm.

Flow rate: 2.0 ml/min
Mobile phase: A) TFA/water (0.1/100, v/v), B) TFA/acetonitrile (0.1/100, v/v)
Gradient: linear gradient from 0% B to 100% B in 2 min then 100% B 2 min

### Detection: UV at 215 nm

### HPLC Condition C:

### Column: YMC-pack ODS-AQ, 50×4.6 mm.

Flow rate: 2.5 ml/min
Mobile phase: A) TFA/water (0.1/100, v/v), B) TFA/acetonitrile (0.1/100, v/v)
Gradient: linear gradient from 10% B to 80% B in 6 min then 80% B 2 min

### Detection: UV at 215 nm

### HPLC Condition D:

### Column: YMC-pack ODS-AQ, 50×4.6 mm.

Flow rate: 3.0 ml/min
Mobile phase: A) TFA/water (0.1/100, v/v), B) TFA/acetonitrile (0.1/100, v/v)
Gradient: linear gradient from 10% B to 80% B in 5 min then 80% B 1.5 min

### Detection: UV at 215 nm

### HPLC Condition E:

### Column: Nucleosil 100-3 C18HD (125×4 mm).

Flow rate: 1.0 ml/min
Mobile phase: A) TFA/water (0.1/100, v/v), B) TFA/acetonitrile (0.1/100, v/v)
Gradient: linear gradient from 2% B to 100% B in 7 min then 100% B 1 min

### Detection: UV at 215 nm

The HPLC conditions A, B, C, D and E can be identified by the subscript prefixes of the T_{Ret} values given in the examples. For instance, B in _{B}t_{Ret}= . . . . Min means condition-B in the case of HPLC.

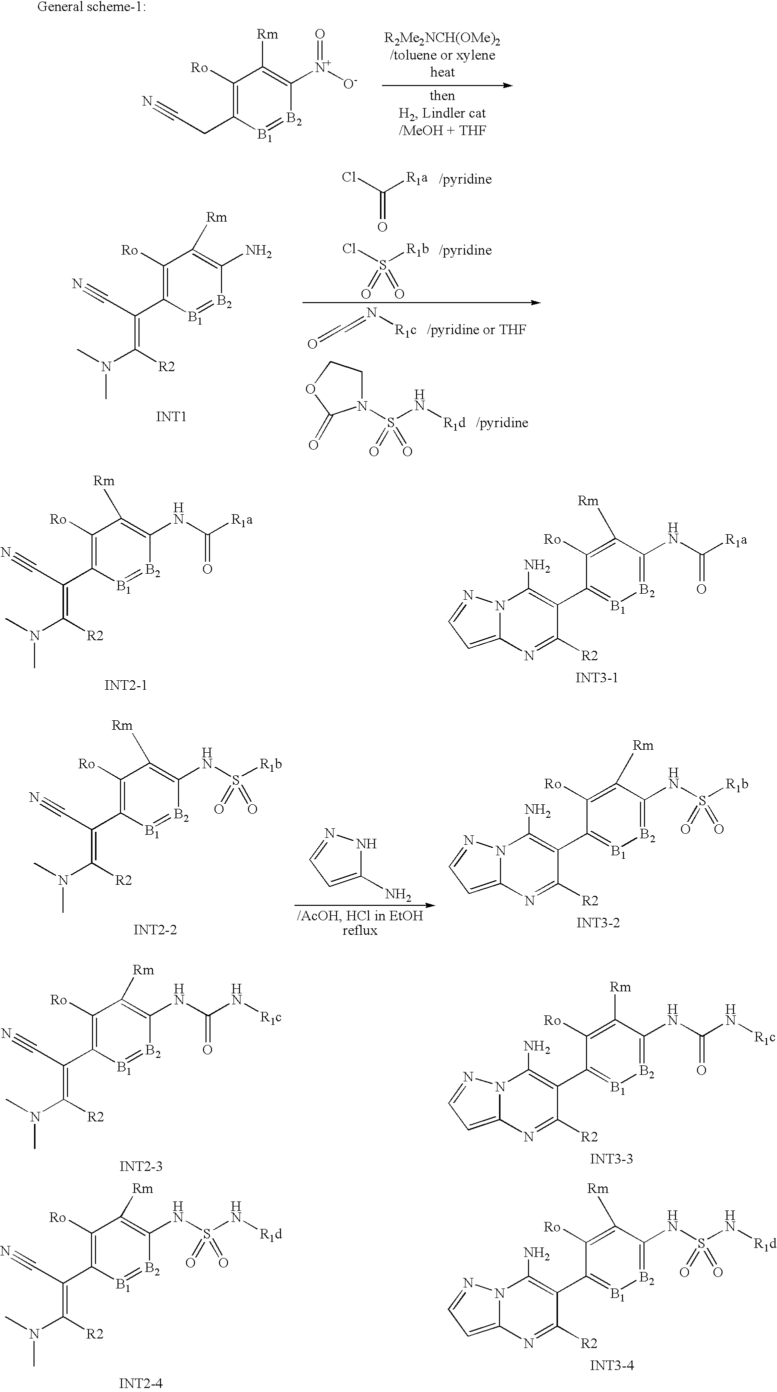

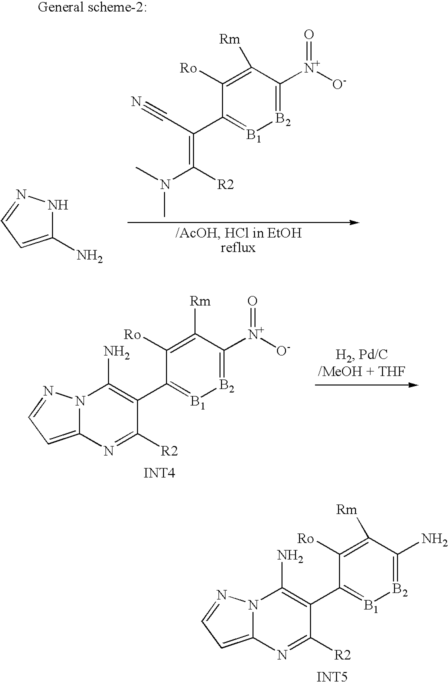

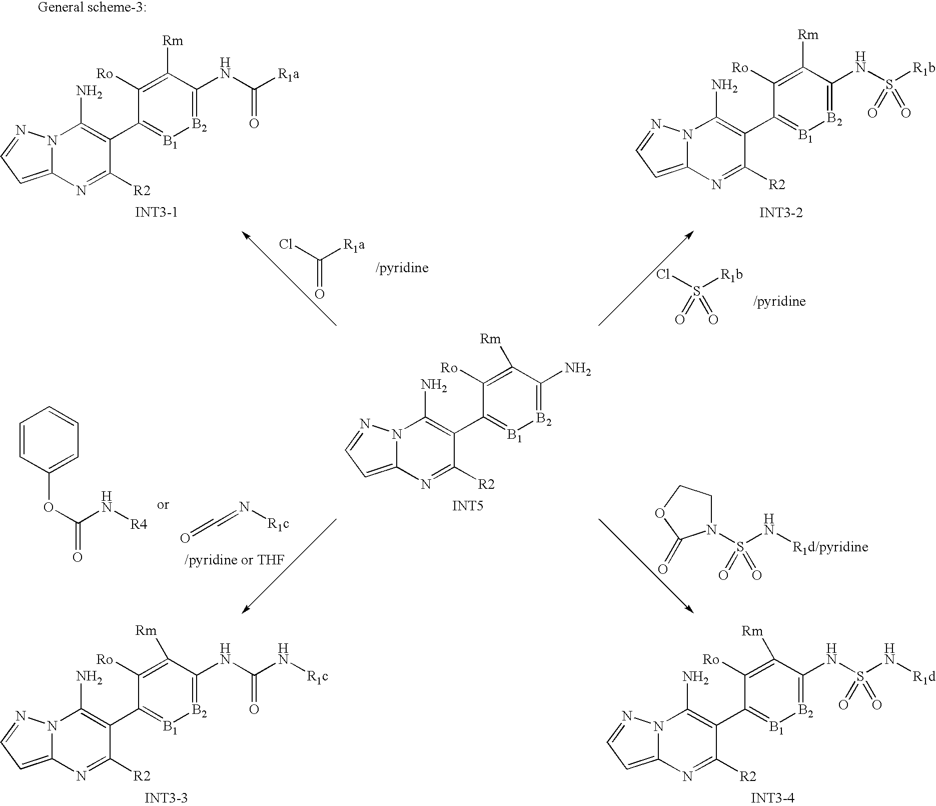

In Schemes 1 to 3, R₁a-C(═O)—, R₁b-S(O)₂—, R₁c-NH—C(═O)— and R₁d-NH—S(O)₂— are corresponding moieties falling under the definition of R1- in formula I, that is, R₁a, R₁b, R₁c and R₁d are moieties that together with the binding groups given form acyl moieties R1. The other moieties are as defined under formula I, preferably as in the Examples.

### Example 1N-[4-(7-Amino-pyrazolo[1,5-a]pyrimidin-6-yl)-phenyl]-2,3-dimethyl-benzenesulfonamide

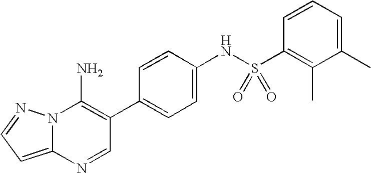

A mixture of 3-aminopyrazole (190 mg, 2.28 mmol) and N-[4-((Z)-1-cyano-2-dimethylamino-vinyl)-phenyl]-2,3-dimethyl-benzenesulfonamide (200 mg, 0.56 mmol) in AcOH (3 mL) and EtOH solution of 1.25M HCl (3 ml) is refluxed for 15 hours. The resulting mixture is concentrated in vacuo, and the product is isolated by filtration, washed with CH₃CN, and dried under reduced pressure to give the title compound as colorless crystal; ES-MS: M+H=394.0; HPLC: _{A}t_{Ret}=3.32 min.

### Intermediate 1.1 N-[4-((Z)-1-Cyano-2-dimethylamino-vinyl)-phenyl]-2,3-dimethyl-benzenesulfonamide

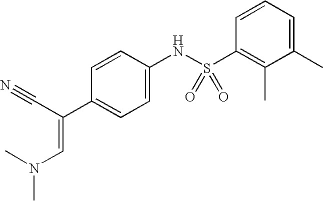

A mixture of (Z)-2-(4-amino-phenyl)-3-dimethylamino-acrylonitrile (1.15 g, 6.14 mmol) and 2,3-dimethylbenzenesulfonyl chloride (1.5 g, 7.33 mmol) (see, WO 2003055478) in pyridine (12 mL) is stirred for 3 hours at room temperature. The resulting mixture is poured into a mixture of ice and water, and the product is isolated by filtration and washed with water, and dried under reduced pressure to give the title compound as yellow powder; ES-MS: M+H=356.1; HPLC: _{A}t_{Ret}=4.48 min.

### Intermediate 1.2 (Z)-2-(4-Amino-phenyl)-3-dimethylamino-acrylonitrile

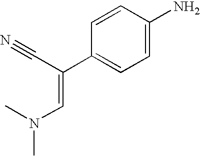

A mixture of (Z)-3-dimethylamino-2-(4-nitro-phenyl)-acrylonitrile (2.0 g, 5.5 mmol) (see, Bulletin des Societes Chimiques Belges (1994), 103(12), 697-703.) and 5% Pd/C (0.1 g) in EtOH (200 mL) and THF (100 mL) is shaken under a H₂-atmosphere (1 bar). After 24 h, the reaction mixture is filtered through Celite and carefully washed with THF. Concentration in vacuo affords the title compound as brown crystal; ES-MS: M+H=188.0; HPLC: _{A}t_{Ret}=1.50 min.

### Example 21-[4-(7-Amino-pyrazolo[1,5-a]pyrimidin-6-yl)-phenyl]-3-[5-tert-butyl-2-(4-fluoro-phenyl)-2H-pyrazol3-yl]-urea

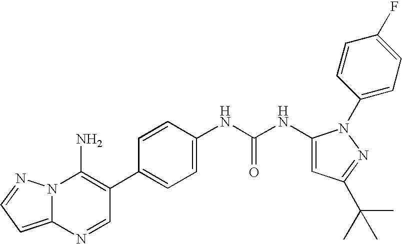

1-[5-tert-Butyl-2-(4-fluoro-phenyl)-2H-pyrazol-3-yl]-3-[4-((Z)-1-cyano-2-dimethylamino-vinyl)phenyl]urea (240 mg, 0.54 mmol) and 3-amino pyrazole (44 mg, 0.54 mmol) are dissolved in HCl/EtOH (1.25 M solution; 6 ml) and stirred at 90° C. for 1 h. The reaction mixture is concentrated, the solid residue is washed with H₂O and EtOAc and dried to give the title compound as a yellow powder. ES-MS: M+H=328.0; Mp 176-178° C.

### Intermediate 2.1 1-[5-tert-Butyl-2-(4-fluoro-phenyl)-2H-pyrazol-3-yl]-3-[4-((Z)-1-cyano-2-dimethylamino-vinyl)-phenyl]urea

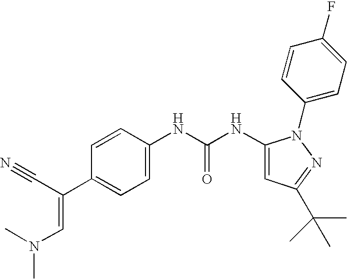

(Z)-2-(4-Amino-phenyl)-3-dimethylamino-acrylonitrile (intermediate 1.2; 161 mg, 0.86 mmol) is dissolved in THF (4 ml) at rt and added to a solution of [5-tert-butyl-2-(4-fluoro-phenyl)-2-H-pyrazol-3-yl]carbamic acid phenyl ester (step 2.2.) in THF (1 ml). The reaction mixture is kept under stirring for 2 h at ambient temperature and then concentrated under reduced pressure. The remaining crude product is purified by flash chromatography (combi-flash, 40 g column, CH₂Cl₂/MeOH, gradient 0-5% MeOH) to give the title compound as a yellow solid. ES-MS: M+H=447.15; HPLC: _{B}t_{Ret}=2.36 min.

### Intermediate 2.2 [5-tert-Butyl-2-(4-fluoro-phenyl)-2-H-pyrazol-3-yl]carbamic Acid Phenyl Ester

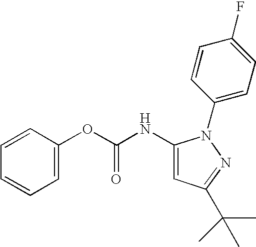

5-tert-Butyl-2-(4-fluoro-phenyl)-2H-pyrazol-3-ylamine (200 mg, 0.86 mmol) is dissolved in THF (5 ml) and treated at 0° C. with phenyl chloro formate (107 μl, 0.86 mmol) and pyridine (69 μl, 0.86 mmol). The reaction mixture is allowed to stir at 0° C. for 20 min. It is then diluted with EtOAc and successively washed with H₂O and brine, dried and concentrated in vacuuo to give the title compound as a yellow oil which is used without further purification for the next step.

### Intermediate 2.3 5-tert-Butyl-2-4-fluoro-phenyl)-2H-pyrazol-3-ylamine

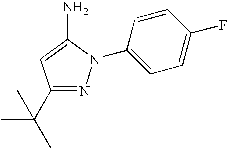

The title compound is prepared according to a published literature procedure (see J. Med. Chem. 2002, 45, 2994-3008.) 4.17 g (32.3 mMol) of pivaloylacetonitrile are added to a solution of 4.20 g (32.3 mMol) 4-fluoro-phenylhydrazine in 150 mL of toluene at rt, and the resulting yellow solution is heated to and kept under reflux for 12 h. After completion, the reaction mixture is concentrated, and the resulting crude product is purified by flash chromatography (SiO₂, 100% CH₂Cl₂) to give the title compound as a yellow solid. MS: [M+1]⁺=234.3; ¹HNMR (CDCl₃) 7.59 (d, 2H), 7.10 (d, 2H), 5.58 (s, 1H), 3.62 (brs, 2H, NH₂), 1.32 (s, 9H).

### Example 3N-[Amino-pyrazolo[1,5-a]pyrimidin-6-yl)-3-methoxy-phenyl]-2,3-dichloro-benzenesulfonamide

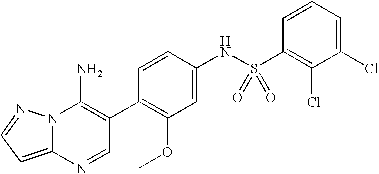

6-(4-Amino-2-methoxy-phenyl)-pyrazolo[1,5-a]pyrimidin-7-ylamine (100 mg, 0.39 mmol) is dissolved in pyridine (4 mL) and 2,3-dichlorobenzene sulfonylchloride (144 mg, 0.58 mmol) is added at rt. The reaction is stirred at ambient temperature for 45 min and then concentrated under reduced pressure. The residual crude product is purified by flash chromatography (combi-flash, 40 g column, CH₂Cl₂/MeOH, gradient 1-8% MeOH) to give the title compound as a yellow solid. ES-MS: M+H=466.92; HPLC: _{B}t_{Ret}=1.93 min, Mp 258-259° C.

### Intermediate 3.1 6-(4-Amino-2-methoxy-phenyl)-pyrazolo[1,5-a]pyrimidin-7-ylamine

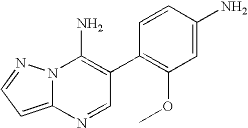

6-(2-Methoxy-4-nitro-phenyl)-pyrazolo[1,5-a]pyrimidin-7-ylamine (2.45 g, 8.6 mmol) is dissolved in THF/MeOH (2:1, 60 mL) and hydrogenated over Raney-Nickel (0.7 g) at ambient pressure and temperature for 14 h. After completion the reaction mixture is filtered over a pad of celite, concentrated and purified by flash chromatography (combi-flash, 40 g column, CH₂Cl₂/MeOH, gradient 0-10% MeOH) to give the title compound as a yellow solid. ES-MS: M+H=256.15; HPLC: _{B}t_{Ret}=1.36 min.

### Intermediate 3.2 6-(2-Methoxy-4-nitro-phenyl)-pyrazolo[1,5-a]pyrimidin-7-ylamine

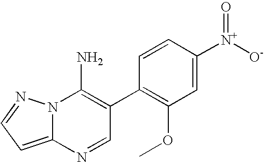

(Z)-3-Dimethylamino-2-(2-methoxy-4-nitro-phenyl)-acrylonitrile (2.7 g, 11 mmol) is dissolved in EtOH (25 mL). 3-Amino pyrazole (907 mg, 11 mmol) is added followed by HCl (1.25 M solution in EtOH, 25 mL). The reaction mixture is then heated to 90° C. and stirred for 2 h. It is allowed to cool again and EtOH is removed under reduced pressure. The residual material is taken up with EtOAc, washed with brine, dried, concentrated and dried under high vacuum to give the crude title compound as a yellow oil which is used for the next step without further purification. M+H=286.17; HPLC: _{B}t_{Ret}=1.64 min.

### Intermediate 3.3 (Z)-3-Dimethylamino-2-(2-methoxy-4-nitro-phenyl)-acrylonitrile

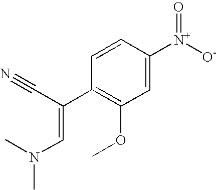

(2-Methoxy-4-nitro-phenyl)-acetonitrile (2.1 g, 11 mmol) is dissolved in toluene (25 mL) and N,N-dimethyl formamide dimethyl acetal (2.9 mL, 22 mmol) is added at rt. The reaction is then heated to 120° C. and stirred for 2.5 h. It is subsequently cooled again and all volatiles are removed under reduced pressure. The remaining crude product is dried under high vacuum to give the title compound as a yellow oil. ES-MS: M+H=248.18; HPLC: _{B}t_{Ret}=2.17 min.

### Intermediate 3.4. (2-Methoxy-4-nitro-phenyl)-acetonitrile

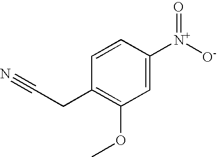

Cyano-(2-methoxy-4-nitro-phenyl)-acetic acid ethyl ester (5.8 g, 22 mmol) is dissolved in EtOH (80 mL) and treated with aqueous 6N HCl solution at rt. The reaction mixture is then heated to 100° C. and stirred for 2 h. It is subsequently cooled to rt and EtOH is removed under reduced pressure. EtOAc is added and the aqueous layer is repeatedly extracted with EtOAc. The combined organic extracts are washed with brine, dried and concentrated. The crude product is purified by flash chromatography (combi-flash, 120 g column, CH₂Cl₂) to give the title compound as a yellow oil. ES-MS: M+H=193.19; HPLC: _{B}t_{Ret}=2.05 min

### Intermediate 3.5. Cyano-(2-methoxy-4-nitro-phenyl)-acetic Acid Ethyl Ester

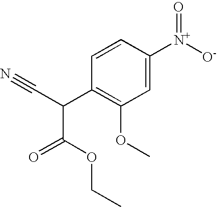

2-Chloro-5-nitroanisole (5.0 g, 26.6 mmol) and cyano acetic acid ethyl ester (4.8 mL, 45 mmol) are dissolved in DMF (60 mL). Solid K₂CO₃ (anhydrous, 6.26 g, 45 mmol) is added at rt and then the reaction mixture is warmed to 120° C. and stirred for 4 h. It is allowed to cool again and DMF is removed under reduced pressure. The residue is taken up in EtOAc and ice-water and carefully neutralized with H₂SO₄. The organic layer is separated, washed with brine, dried and concentrated. To give the crude title compound which was used without further purification for the next step. ES-MS: M+H=265.20; HPLC: _{B}t_{Ret}=2.18 min.

### Example 4

### N-[4-(7-amino-5-methyl-pyrazolo[1,5-a]pyrimidin-6-yl)-phenyl]-2,3-dichloro-benzenesulfonamide

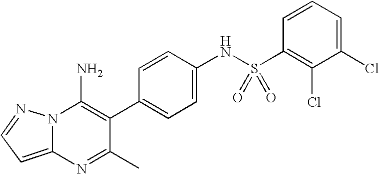

A mixture of 3-aminopyrazole (39 mg, 0.47 mmol) and 2,3-dichloro-N-[4-(1-cyano-2-oxopropyl)-phenyl]-benzenesulfonamide (150 mg, 0.39 mmol) in AcOH (2 mL) and EtOH solution of 1.25M HCl (2 ml) is refluxed for 11.5 hours. The resulting mixture is concentrated in vacuo, and the product is isolated by filtration and washed with CH₃CN, and dried under reduced pressure to give the compound given in the formula immediately above as colorless crystal; ES-MS: M+H=449.9; HPLC: _{A}t_{Ret}=3.55 min.

### Intermediate 4.1 2,3-Dichloro-N-[4-(1-cyano-2-oxo-propyl)-phenyl]-benzenesulfonamide

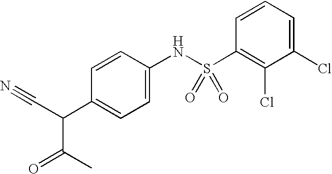

A mixture of 2,3-dichloro-N-(4-cyanomethyl-phenyl)-benzenesulfonamide (200 mg, 0.59 mmol) and sodium methoxide (236 mg, 1.77 mmol) in acetic acid butyl ester (1.2 mL, 27.2 mmol) is stirred at 80° C. for 1 h. The residual material is taken up with EtOAc. The combined organic extracts are washed with brine, dried and concentrated. The crude product is purified by flash chromatography to give the title compound as a yellow oil. ES-MS: M+H=382.9; HPLC: _{A}t_{Ret}=4.24 min

### Intermediate 4.2 2,3-Dichloro-N-(4-cyanomethyl-phenyl)-benzenesulfonamide

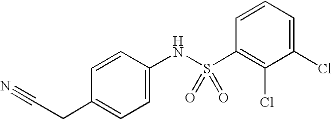

A solution of (4-amino-phenyl)-acetonitrile (3.0 g, 22.7 mmol) and 2,3-dichlorobenzenesulfonyl chloride (6.7 g, 27.2 mmol) in pyridine (114 mL) is stirred for 30 min at room temperature. The resulting mixture is poured into a mixture of ice and water, and the product is isolated by filtration and washed with water, and dried under reduced pressure to give the title compound as yellow powder; ES-MS: M+H=340.9; HPLC: _{A}t_{Ret}=4.33 min.

TABLE

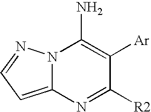

| | | | Analytical data |
|---|---|---|---|
| Example | R2 | Ar | MS/HPLC/m.p |
| 5 | H | 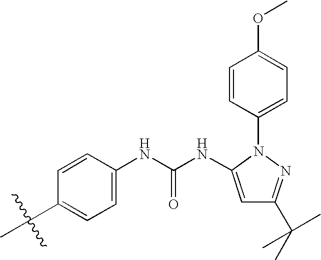 | M + H = 497.2 182-183° C. |
| 6 | H | 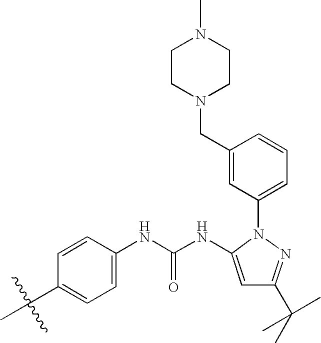 | M + H = 579.8 140-142° C. |
| 7 | H | 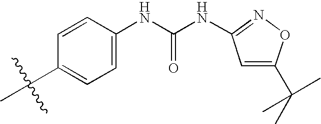 | M + H = 392.5 201-202° C. |
| 8 | H | 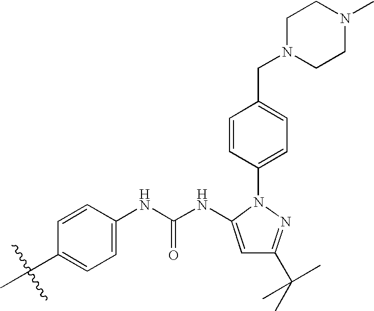 | M + H = 579.8 154-156° C. |
| 9 | H | 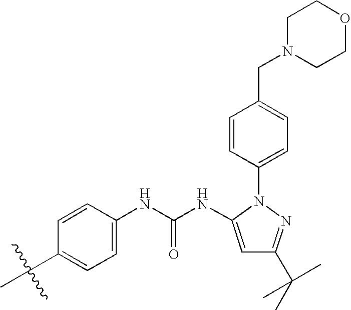 | M + H = 566.1 244-245° C. |
| 10 | H | 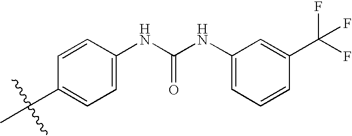 | M + H = 413.1 _{D}t_{Ret} = 5.31 min |
| 11 | H | 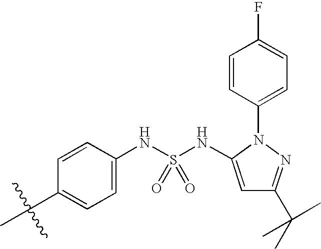 | M + H = 521.0 _{A}t_{Ret} = 3.70 min |
| 12 | H | 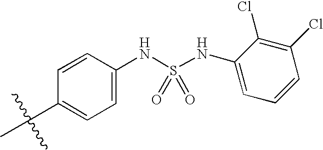 | M + H = 450.9 _{A}t_{Ret} = 3.41 min |
| 13 | H | 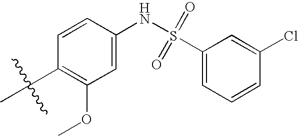 | M + H = 431.9 127-129° C. |
| 14 | H | 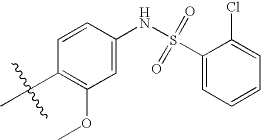 | M + H = 431.9 149-150° C. |
| 15 | H | 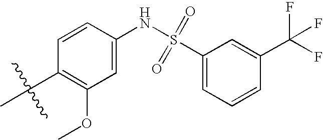 | M + H = 464.9 122-123° C. |
| 16 | H | 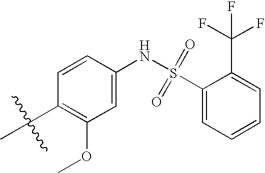 | M + H = 465.0 151-152° C. |
| 17 | H | 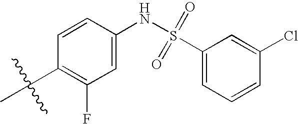 | M + H = 418.9 274-276° C. |
| 18 | H | 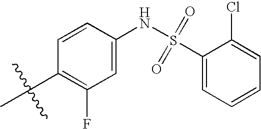 | M + H = 419.0 260° C. (decomposition) |
| 19 | H | 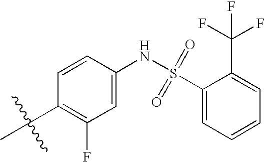 | M + H = 452.9 233-234° C. |
| 20 | H | 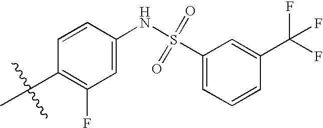 | M + H = 452.9 294-295° C. |
| 21 | H | 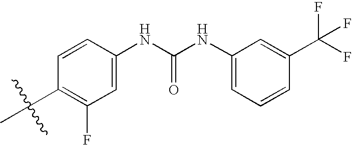 | M + H = 432.0 146-148° C. |
| 22 | H | 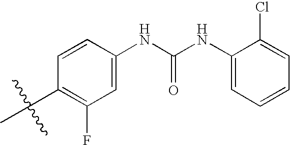 | M + H = 397.0 223-224° C. |
| 23 | H | 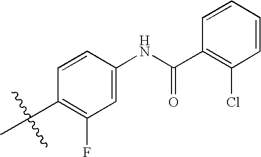 | M + H = 383.0 135-136° C. |
| 24 | H | 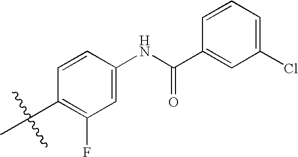 | M + H = 383.0 302-303° C. |
| 25 | H | 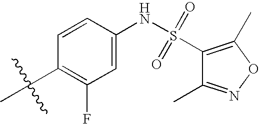 | M + H = 403.8 269-270° C. |
| 26 | H | 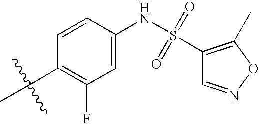 | M+ = 388.8 210-211° C. |
| 27 | H | 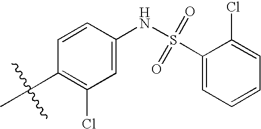 | M + H = 435.9 258-260° C. |
| 28 | H | 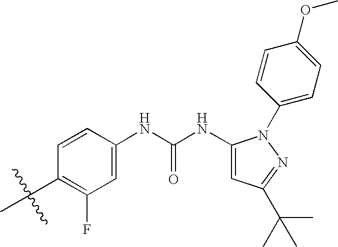 | M+ = 514.8 149-150° C. |
| 29 | H | 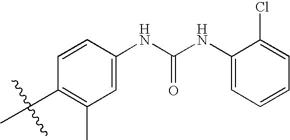 | M + H = 393.0 _{A}t_{Ret} = 3.53 min |
| 30 | H | 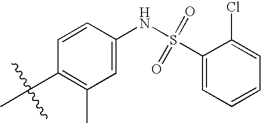 | M + H = 314.0 _{A}t_{Ret} = 3.19 min |
| 31 | H | 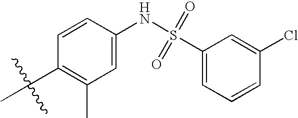 | M + H = 414.0 _{A}t_{Ret} = 3.42 min |
| 32 | H | 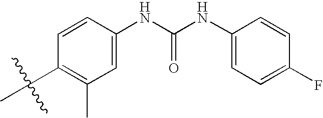 | M + H = 377.1 _{A}t_{Ret} = 3.29 min |
| 33 | H | 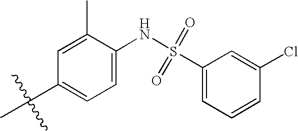 | M+ = 414.0 _{A}t_{Ret} = 3.43 min |
| 34 | H | 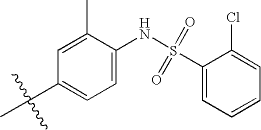 | M+ = 414.0 _{A}t_{Ret} = 3.25 min |
| 35 | H | 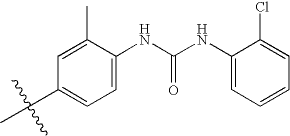 | M+ = 393.0 _{A}t_{Ret} = 3.49 min |
| 36 | H | 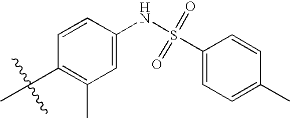 | M + H = 394.1 _{C}t_{Ret} = 4.77 min |
| 37 | H | 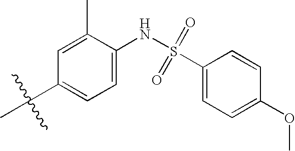 | M + H = 410.1 _{C}t_{Ret} = 4.61 min |
| 38 | H | 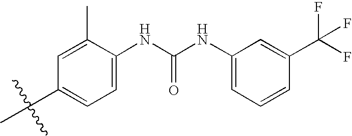 | _{D}t_{Ret} = 5.27 min 319-323° C. |
| 39 | Me | 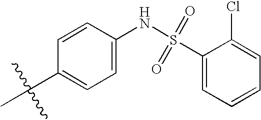 | M + H = 414.0 _{A}t_{Ret} = 3.20 min |
| 40 | Me | 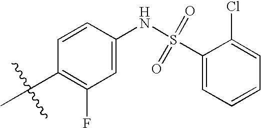 | M + H = 432.0 _{A}t_{Ret} = 3.30 min |
| 41 | H | 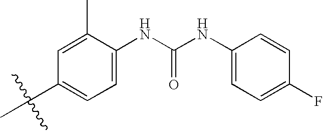 | M + H = 377.1 _{A}t_{Ret} = 3.28 min |
| 42 | H | 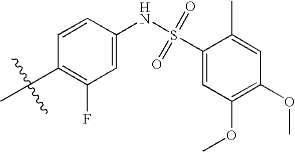 | M + H = 458.83 244-245° C. |
| 43 | H | 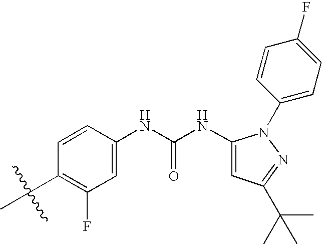 | M + H = 503.67 211-212° C. |
| 44 | Me | 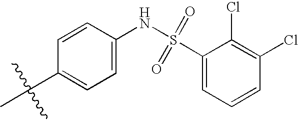 | M + H = 449.9 _{A}t_{Ret} = 3.55 min |
| 45 | H | 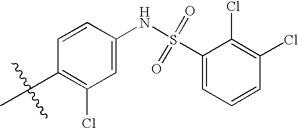 | M + H = 469.80° C. |
| 46 | Me | 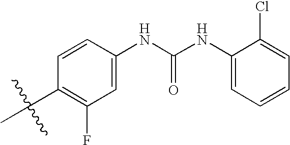 | M + H = 411.83 160-161° C. |
| 47 | Me | 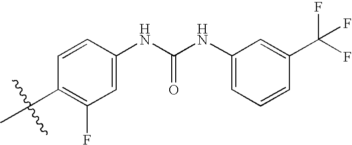 | M + H = 445.80 154-156° C. |
| 48 | Me | 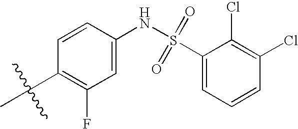 | M + H = 468.70 254-255° C. |
| 49 | Me | 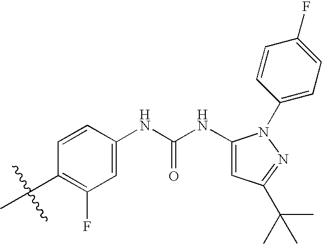 | M+ = 516.86 164-165° C. |
| 50 | H | 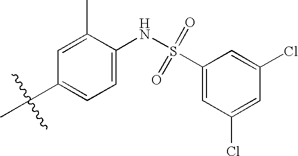 | M+ = 448.0 _{C}t_{Ret} = 4.00 min |
| 51 | H | 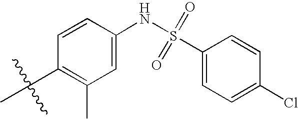 | M + H = 414.0 _{C}t_{Ret} = 4.74 min |
| 52 | H | 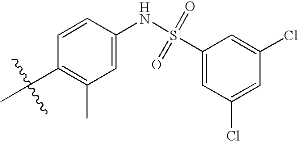 | M + H = 449.2 _{C}t_{Ret} = 4.26 min |
| 53 | H | 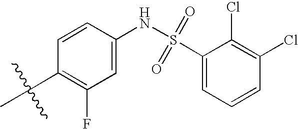 | M + H = 453.0 248-250° C. |
| 54 | Me | 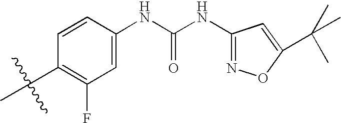 | M + H = 424.89 156-157° C. |
| 55 | H | 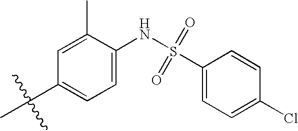 | M + H = 414.0 _{C}t_{Ret} = 4.06 min |
| 56 | H | 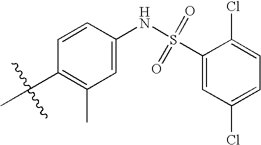 | M+ = 448.2 _{C}t_{Ret} = 4.42 min |
| 57 | H | 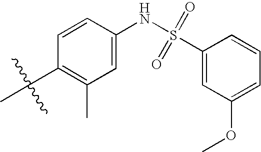 | M + H = 410.1 _{C}t_{Ret} = 3.52 min |
| 58 | H | 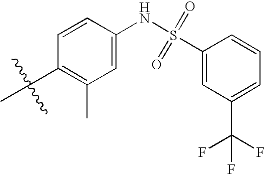 | M + H = 448.1 _{C}t_{Ret} = 3.64 min |
| 59 | H | 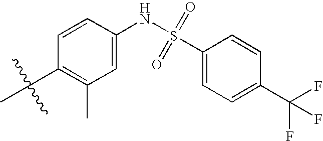 | M + H = 448.1 _{C}t_{Ret} = 3.65 min |
| 60 | H | 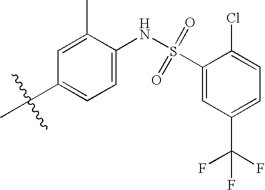 | M + H = 482.0 _{C}t_{Ret} = 4.47 min |
| 61 | H | 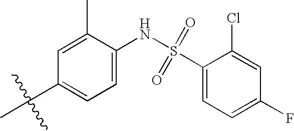 | M + H = 432.0 _{C}t_{Ret} = 3.93 min |
| 62 | H | 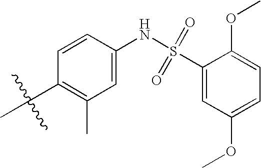 | M + H = 440.1 _{C}t_{Ret} = 3.39 min |
| 63 | H | 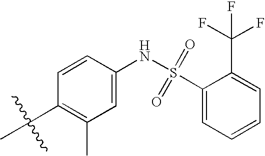 | M + H = 448.0 _{C}t_{Ret} = 3.28 min |
| 64 | H | 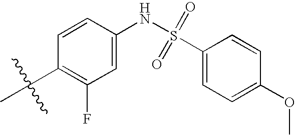 | M + H = 414.1 _{C}t_{Ret} = 3.57 min |
| 65 | H | 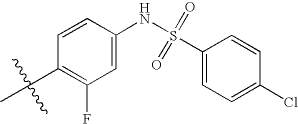 | M + H = 418.0 _{C}t_{Ret} = 3.43 min |
| 66 | H | 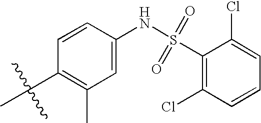 | M+ = 448.0 _{C}t_{Ret} = 3.40 min |
| 67 | H | 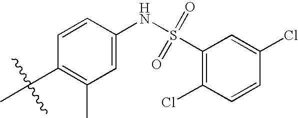 | M+ = 448.0 _{C}t_{Ret} = 4.86 min |
| 68 | H | 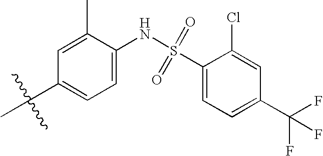 | M+ = 482.0 _{C}t_{Ret} = 3.91 min |
| 69 | H | 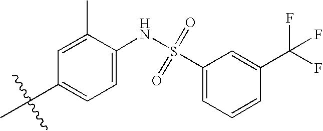 | M + H = 448.0 _{C}t_{Ret} = 3.64 min |
| 70 | H | 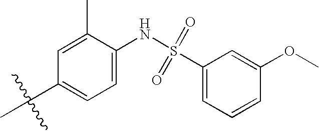 | M + H = 410.1 _{C}t_{Ret} = 3.15 min |
| 71 | H | 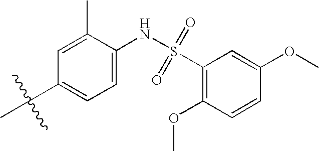 | M + H = 440.0 _{C}t_{Ret} = 3.13 min |
| 72 | H | 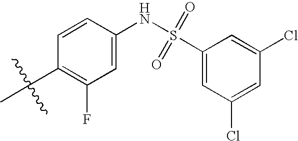 | M+ = 452.0 _{C}t_{Ret} = 4.44 min |
| 73 | H | 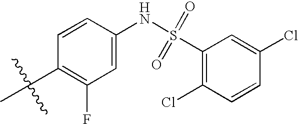 | M+ = 452.0 _{C}t_{Ret} = 3.48 min |
| 74 | H | 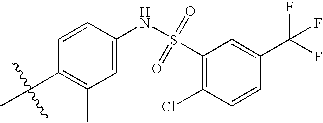 | M + H = 482.1 _{C}t_{Ret} = 3.82 min |
| 75 | H | 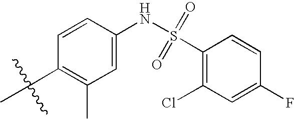 | M + H = 432.1 _{C}t_{Ret} = 3.66 min |
| 76 | H | 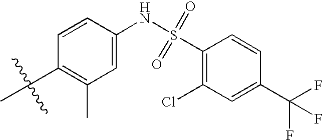 | M+ = 482.0 _{C}t_{Ret} = 5.11 min |
| 77 | H | 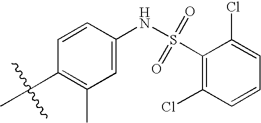 | M+ = 448.0 _{C}t_{Ret} = 3.35 min |
| 78 | H | 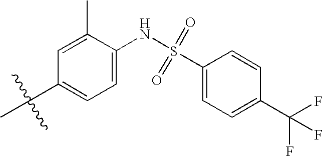 | M + H = 448.0 _{C}t_{Ret} = 4.93 min |
| 79 | H | 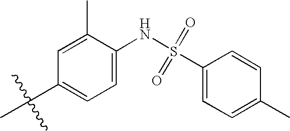 | M + H = 394.1 _{C}t_{Ret} = 3.34 min |
| 80 | H | 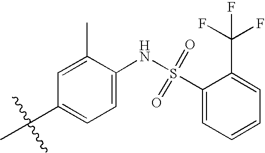 | M+ = 448.0 _{C}t_{Ret} = 3.65 min |
| 81 | H | 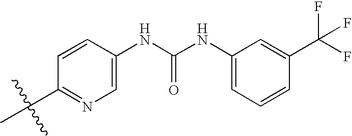 | _{D}t_{Ret} = 5.29 min >360° C. (decomposition) |
| 82 | Me | 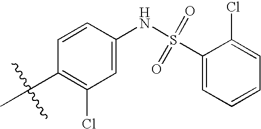 | M + H = 449.2 137-139° C. |
| 83 | H | 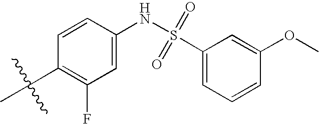 | M + H = 414.1 _{C}t_{Ret} = 3.21 min |
| 84 | H | 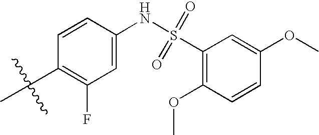 | M + H = 444.1 _{C}t_{Ret} = 3.17 min |
| 85 | H | 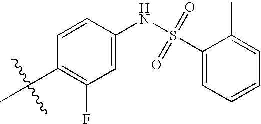 | M + H = 398.1 _{C}t_{Ret} = 3.81 min |
| 86 | H | 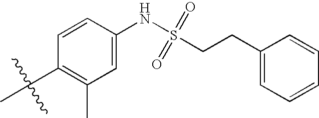 | M + H = 408.1 _{C}t_{Ret} = 8.38 min |
| 87 | H | 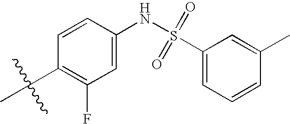 | M + H = 398.1 _{C}t_{Ret} = 3.33 min |
| 88 | H | 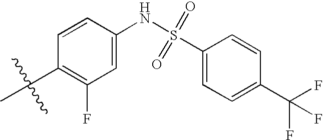 | M − H = 450.3 _{C}t_{Ret} = 4.28 min |
| 89 | H | 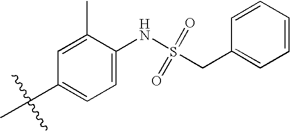 | M + H = 394.0 _{C}t_{Ret} = 2.39 min |
| 90 | H | 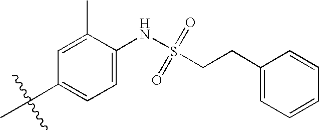 | M + H = 408.1 _{C}t_{Ret} = 2.65 min |
| 91 | H | 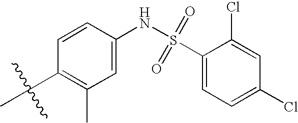 | M+ = 448.0 _{C}t_{Ret} = 3.00 min |
| 92 | H | 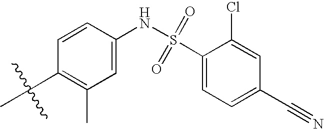 | M+ = 439.0 _{C}t_{Ret} = 2.68 min |
| 93 | H | 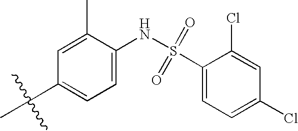 | M+ = 448.0 _{C}t_{Ret} = 3.45 min |
| 94 | H | 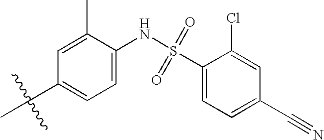 | M+ = 439.0 _{C}t_{Ret} = 2.51 min |
| 95 | H | 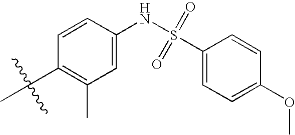 | M + H = 410.1 _{C}t_{Ret} = 3.74 min |
| 96 | H | 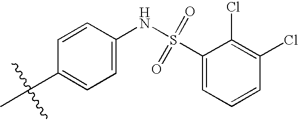 | M + H = 435.3 298-299° C. |
| 97 | H | 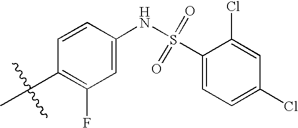 | M+ = 452.0 _{C}t_{Ret} = 3.64 min |
| 98 | H | 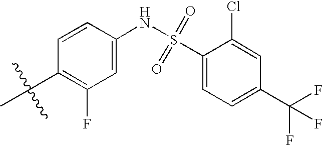 | M+ = 486.1 _{C}t_{Ret} = 3.21 min |
| 99 | H | 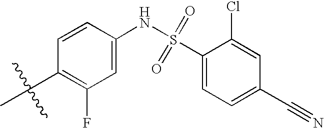 | M + H = 443.0 _{C}t_{Ret} = 2.40 min |
| 100 | H | 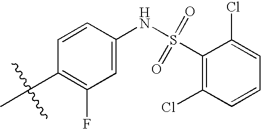 | M+ = 452.0 _{C}t_{Ret} = 2.59 min |
| 101 | H | 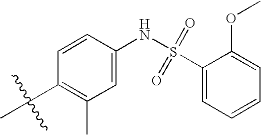 | M + H = 410.1 _{C}t_{Ret} = 2.89 min |
| 102 | H | 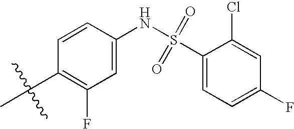 | M + H = 436.1 _{C}t_{Ret} = 3.32 min |
| 103 | H | 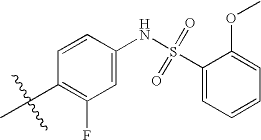 | M + H = 414.1 _{C}t_{Ret} = 2.27 min |
| 104 | H | 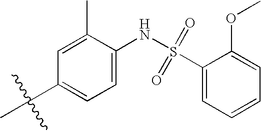 | M + H = 410.1 _{C}t_{Ret} = 2.23 min |
| 105 | 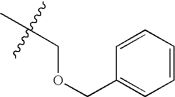 | 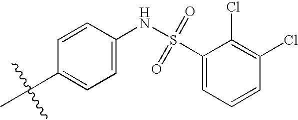 | M − H = 554.8 _{A}t_{Ret} = 4.26 min |

### Example 106

### Soft Capsules

soft gelatin capsules, each comprising as active ingredient 0.05 g of one of the compounds of formula I mentioned in any one of the preceding examples, are prepared as follows:

Composition

| | |
|---|---|
| Active ingredient | 250 g |
| Lauroglycol | 2 litres |

Preparation process: The pulverized active ingredient is suspended in Lauroglykol* (propylene glycol laurate, Gattefossé S. A., Saint Priest, France) and ground in a wet pulverizer to produce a particle size of about 1 to 3 μm. 0.419 g portions of the mixture are then introduced into soft gelatin capsules using a capsule-filling machine.

### Example 107

### Tablets Comprising Compounds of the Formula I

Tablets, comprising, as active ingredient, 100 mg of any one of the compounds of formula I of Examples 1 to 132 are prepared with the following composition, following standard procedures:

Composition

| | | |
|---|---|---|
| Active Ingredient | 100 | mg |
| crystalline lactose | 240 | mg |
| Avicel | 80 | mg |
| PVPPXL | 20 | mg |
| Aerosil | 2 | mg |
| magnesium stearate | 5 | mg |
| | 447 | mg |

Manufacture: The active ingredient is mixed with the carrier materials and compressed by means of a tabletting machine (Korsch EKO, Stempeldurchmesser 10 mm). Avicel® is microcrystalline cellulose (FMC, Philadelphia, USA). PVPPXL is polyvinylpolypyrrolidone, cross-linked (BASF, Germany). Aerosil® is silicium dioxide (Degussa, Germany).

1. A compound of the formula I, 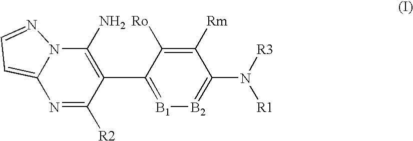
wherein
R1 is acyl,
R2 is hydrogen, lower alkyl, heterocyclyl-lower alkyl wherein heterocyclyl is unsubstituted or substituted and has 3 to 14 ring atoms, hydroxyl-lower alkyl, esterified or etherified hydroxyl-lower alkyl or unsubstituted or substituted amino-lower alkyl;
R3 is hydrogen or unsubstituted or substituted lower alkyl;
B₁ is N or CRo;
B₂ is N or CRm;
and each Ro and Rm, independently of the others, is selected from hydrogen, lower alkyl, halo and lower alkoxy;
with the proviso that if R1 is trifluoromethylphenyl-aminocarbonyl, then R2 is lower alkyl, heterocyclyl-lower alkyl, hydroxyl-lower alkyl, esterified or etherified hydroxyl-lower alkyl or unsubstituted or substituted amino-lower alkyl (that is over than hydrogen) and/or R3 is unsubstituted or substituted lower alkyl (that is other than hydrogen);
or a salt thereof.

2. A compound of the formula I according to claim 1, wherein
R1 unsubstituted or substituted heterocyclylaminocarbonyl wherein heterocyclyl has 3 to 14 ring atoms, unsubstituted or substituted C₆-C₁₄-arylaminosulfonyl, unsubstituted or substituted heterocyclylaminosulfonyl wherein heterocyclyl has 3 to 14 ring atoms, unsubstituted or substituted lower-alkanesulfonyl, unsubstituted or substituted C₆-C₁₄-arylsulfonyl, unsubstituted or substituted heterocyclylsulfonyl wherein heterocyclyl has 3 to 14 ring atoms, or unsubstituted or substituted C₆-C₁₄-arylcarbonyl;
R2 is hydrogen, lower alkyl, heterocyclyl-lower alkyl wherein heterocyclyl is unsubstituted or substituted and has 3 to 14 ring atoms, hydroxyl-lower alkyl, esterified or etherified hydroxyl lower alkyl or unsubstituted or substituted amino-lower alkyl;
R3 is hydrogen or unsubstituted or substituted lower alkyl,
B₁ is N or CRo;
B₂ is N or CRm;
and each Ro and Rm, independently of the others, is selected from hydrogen, lower alkyl, halo and lower alkoxy;
or a (preferably pharmaceutically acceptable) salt thereof.

3. A compound of the formula I according to claim 1 wherein R1 is substituted C₆-C₁₄-arylaminocarbonyl wherein the substituents are selected from C₁-C₇-alkyl, hydroxy-C₁-C₇-alkyl, C₁-C₇-alkoxy-C₁-C₇-alkyl, amino-C₁-C₇-alkyl, N-mono- or N,N-di-(C₁-C₇-alkyl and/or mono-C₁-C₇-alkoxy-C₁-C₇-alkyl and/or (mono- or di-(C₁-C₇-alkyl)-amino-C₁-C₇-alkyl)-amino-C₁-C₇alkyl, lower alkoxy, cyano and preferably halo, especially fluoro, chloro (which is most preferred) or bromo, hydroxy, C₁-C₇-alkoxy, phenyl-C₁-C₇-alkoxy wherein phenyl is unsubstituted or substituted by C₁-C₇-alkoxy and/or halo;
and the other moieties R₂, R₃, B₁, B₂, Ro and Rm are as defined in claim 1;
or a (preferably pharmaceutically acceptable) salt thereof.

4. A compound of the formula I according to claim 1 wherein
R1 is phenylaminocarbonyl wherein phenyl is unsubstituted or substituted by one or more moieties independently selected from lower alkyl, halo (very preferred), especially chloro;
lower alkoxy and cyano:
pyrazolyl-aminocarbonyl or isoxazolylaminocarbonyl where pyrazolyl or isoxazolyl is unsubstituted or substituted by one or two moieties independently selected from the group consisting of lower alkyl and phenyl that is unsubstituted or substituted with halo, lower alkoxy, piperazino-lower alkyl, 4-lower alkylpiperazino-lower alkyl and morpholino-lower alkyl;
pyrazoyl-aminosulfonyl or isoxazolyaminosulfonyl, where each pyrazolyl or isoxazolyl is unsubstituted or substituted by one or two moieties independently selected from the group consisting of lower alkyl and phenyl that is unsubstituted or substituted with halo, lower alkoxy, piperazino-lower alkyl, 4-lower alkylpiperazino-lower alkyl and morpholino-lower alkyl;
phenyl-lower alkanesulfonyl, wherein phenyl is unsubstituted (preferred) or substituted with one or more, e.g. up to three, moieties independently selected from the group consisting of lower alkyl, halo (especially preferred), halo-lower alkyl, lower alkoxy and cyano;
phenylsulfonyl wherein the phenyl is unsubstituted or substituted by one or more moieties independently selected from the group consisting of lower alkyl, halo (preferred) halo-lower alkyl, lower alkoxy and cyano;
R2 is hydrogen, lower alkyl, especially methyl, piperazino-lower alkyl, especially piperazinomethyl, 4-lower alkyl-piperazino-lower alkyl, especially 4-methyl-piperazinomethyl, hydroxyl-lower alkyl, especially hydroxylmethyl, lower alkoxy-lower alkyl, especially lower-alkoxymethyl or phenyl-lower alkoxy-lower alkyl, especially benzyloxymethyl;
R3 is hydrogen (preferred) or lower alkyl,
B₁ is N or CRo;
B₂ is CRm;
and each Ro and Rm, independently of the others, is selected from hydrogen, lower alkyl, especially methyl, halo, especially fluoro or chloro, and lower alkoxy, especially methoxy;
or a (preferably pharmaceutically acceptable) salt thereof.

5. A compound of the formula I according to claim 1 wherein
R1 is unsubstituted or substituted heterocyclylaminocarbonyl wherein heterocyclyl has 3 to 14 ring atoms;
R2 is hydrogen lower alkyl, heterocyclyl-lower-alkyl wherein heterocyclyl is unsubstituted, or substituted and has 3 to 14 ring atoms, hydroxyl-lower alkyl, especially hydroxylmethyl, acyloxy-lower alkyl, especially lower alkanoyloxymethyl, unsubstituted or substituted lower alkoxy-lower alkyl, especially lower-alkoxymethyl or phenyl-lower alkoxymethyl, or unsubstituted or substituted amino-lower alkyl, especially aminomethyl or N-mono- or N,N-di-(lower alkyl and/or phenyl lower alkyl)-amino-methyl;
R3 is hydrogen or unsubstituted or substituted lower alkyl;
B₁ is N or CRo;
B₂ is N or CRm;
and each Ro and Rm, independently of the others, is selected from hydrogen, lower alkyl, halo and lower alkoxy;
or a (preferably pharmaceutically acceptable) salt thereof.

6. A compound of the formula I according to claim 1, selected from the group of compounds with the names
N-[4-(7-amino-pyrazolo[1,5-a]pyrimidin-6-yl)-phenyl]-2,3-dimethyl-benzenesulfonamide
1-[4-(7-amino-pyrazolo[1,5-a]pyrimidin-6-yl)-phenyl]-3-[5-tert-butyl-2-(4-fluoro)-phenyl)-2H-pyrazol3-yl]-urea
N-[amino-pyrazolo[1,5-a]pyrimidin-6-yl)-3-methoxy-phenyl]-2,3-dichloro-benzenesulfonamide
and
N-[4-(7-amino-5-methyl-pyrazolo[1,5-a]pyrimidin-6-yl)-phenyl]-2,3-dichloro-benzenesulfonamide,
or a (preferably pharmaceutically acceptable) salt thereof.

7. A compound of the formula I according to claim 1, selected from the group of compounds represented in the following table:

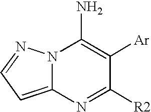

| Compound | R2 | Ar |
|---|---|---|
| 5 | H | 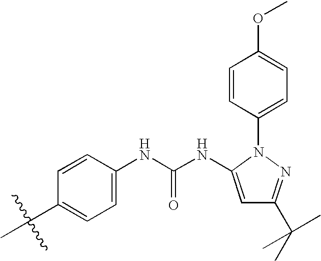 |
| 6 | H | 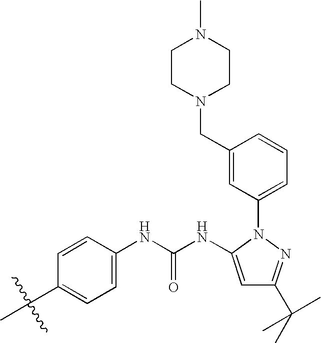 |
| 7 | H | 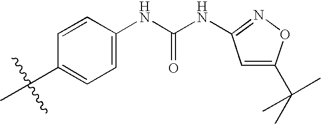 |
| 8 | H | 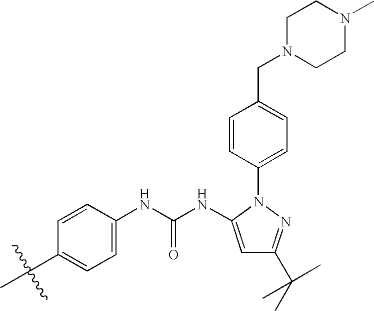 |
| 9 | H | 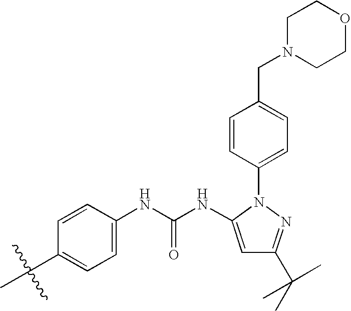 |
| 10 | H | 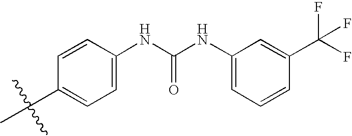 |
| 11 | H | 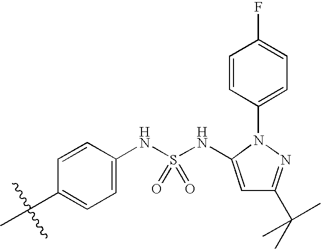 |
| 12 | H | 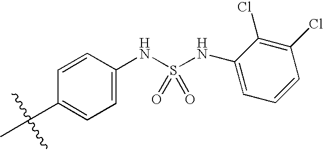 |
| 13 | H | 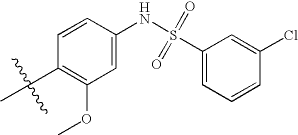 |
| 14 | H | 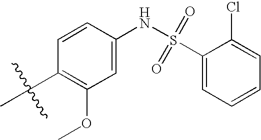 |
| 15 | H | 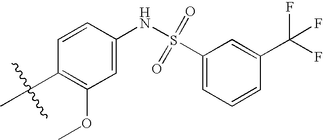 |
| 16 | H | 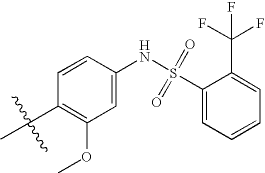 |
| 17 | H | 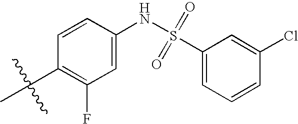 |
| 18 | H | 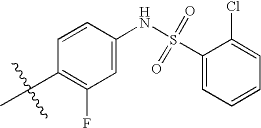 |
| 19 | H | 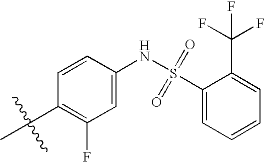 |
| 20 | H | 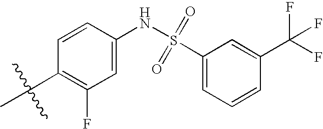 |
| 21 | H | 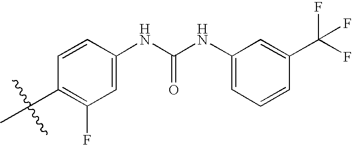 |
| 22 | H | 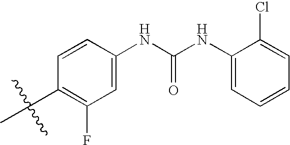 |
| 23 | H | 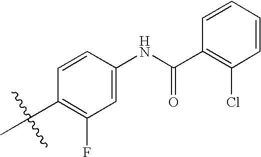 |
| 24 | H | 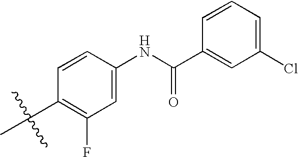 |
| 25 | H | 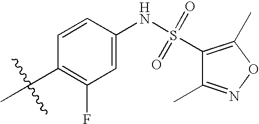 |
| 26 | H | 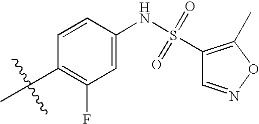 |
| 27 | H | 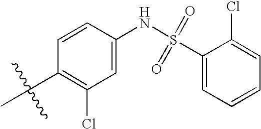 |
| 28 | H | 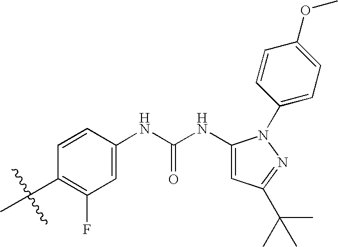 |
| 29 | H | 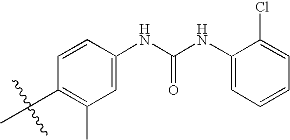 |
| 30 | H | 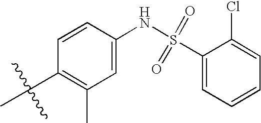 |
| 31 | H | 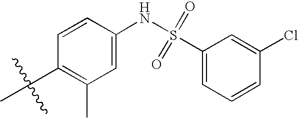 |
| 32 | H | 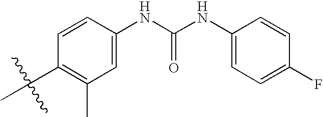 |
| 33 | H | 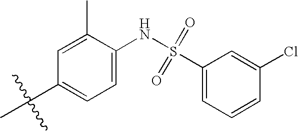 |
| 34 | H | 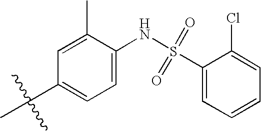 |
| 35 | H | 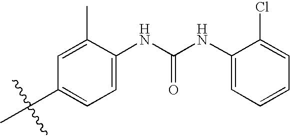 |
| 36 | H | 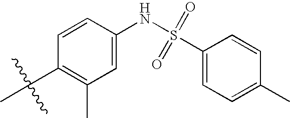 |
| 37 | H | 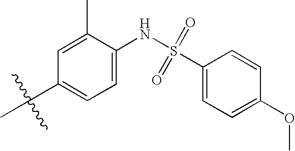 |
| 38 | H | 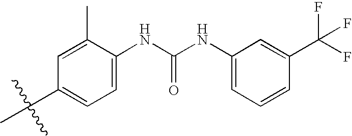 |
| 39 | Me | 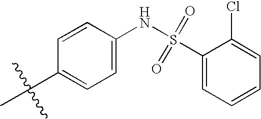 |
| 40 | Me | 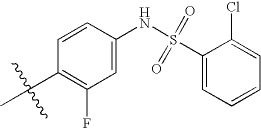 |
| 41 | H | 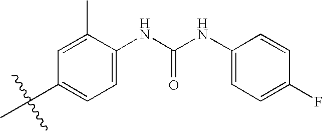 |
| 42 | H | 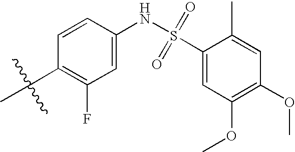 |
| 43 | H | 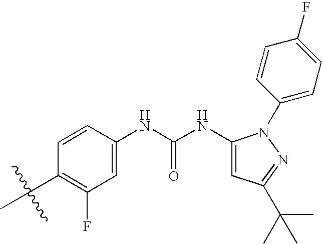 |
| 44 | Me | 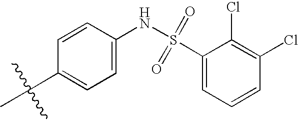 |
| 45 | H | 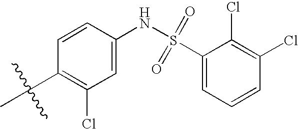 |
| 46 | Me | 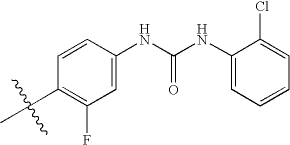 |
| 47 | Me | 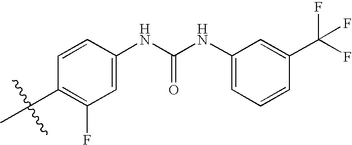 |
| 48 | Me | 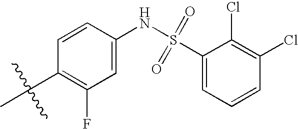 |
| 49 | Me | 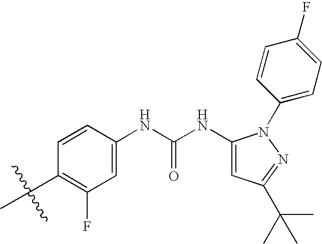 |
| 50 | H | 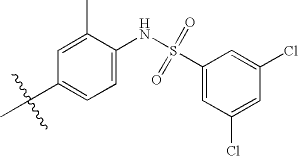 |
| 51 | H | 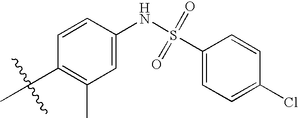 |
| 52 | H | 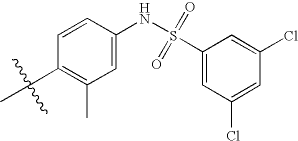 |
| 53 | H | 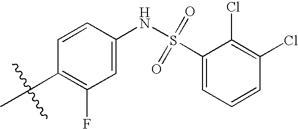 |
| 54 | Me | 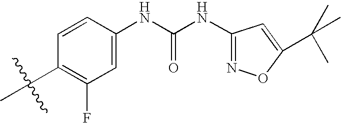 |
| 55 | H | 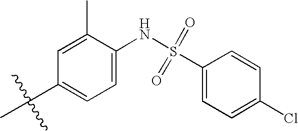 |
| 56 | H | 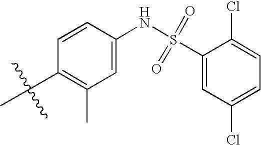 |
| 57 | H | 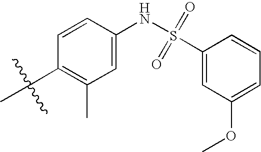 |
| 58 | H | 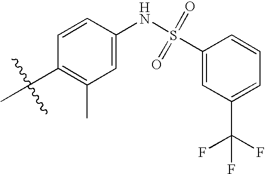 |
| 59 | H | 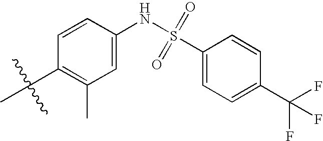 |
| 60 | H | 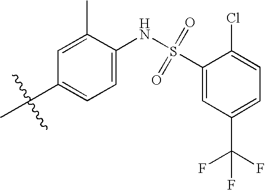 |
| 61 | H | 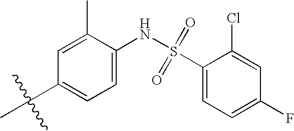 |
| 62 | H | 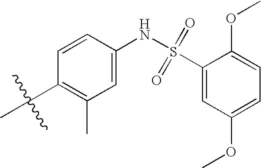 |
| 63 | H | 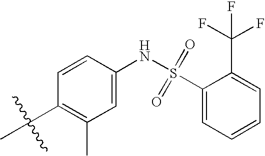 |
| 64 | H | 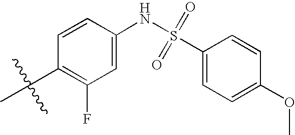 |
| 65 | H | 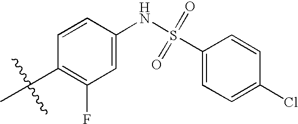 |
| 66 | H | 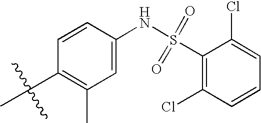 |
| 67 | H | 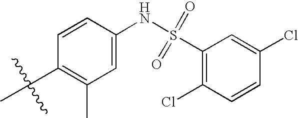 |
| 68 | H | 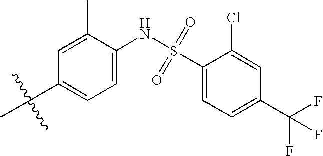 |
| 69 | H | 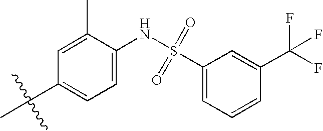 |
| 70 | H | 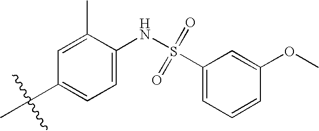 |
| 71 | H | 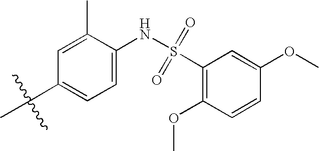 |
| 72 | H | 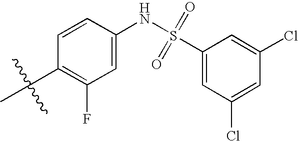 |
| 73 | H | 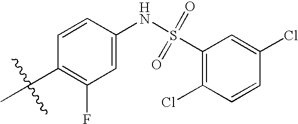 |
| 74 | H | 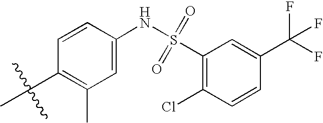 |
| 75 | H | 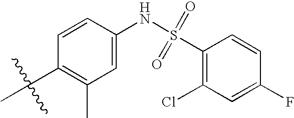 |
| 76 | H | 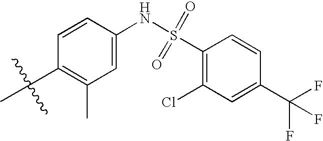 |
| 77 | H | 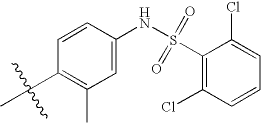 |
| 78 | H | 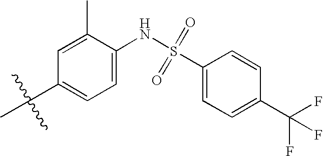 |
| 79 | H | 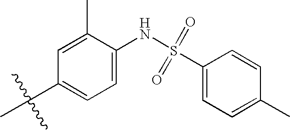 |
| 80 | H | 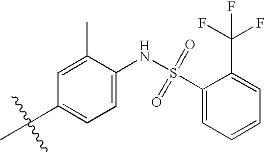 |
| 81 | H | 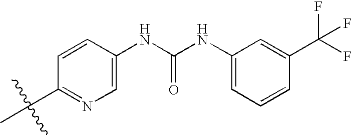 |
| 82 | Me | 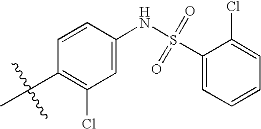 |
| 83 | H | 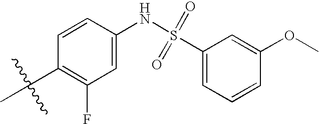 |
| 84 | H | 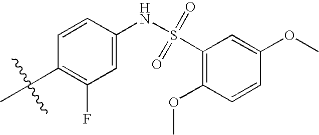 |
| 85 | H | 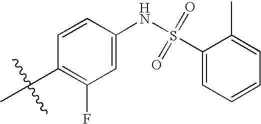 |
| 86 | H | 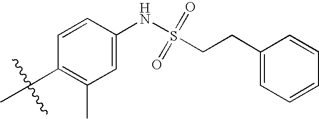 |
| 87 | H | 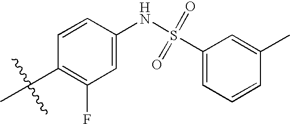 |
| 88 | H | 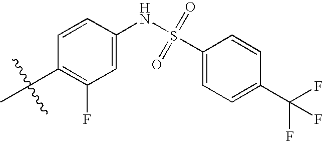 |
| 89 | H | 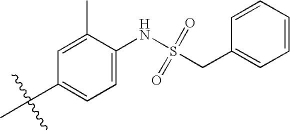 |
| 90 | H | 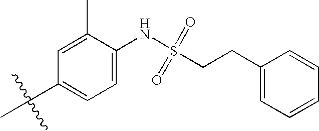 |
| 91 | H | 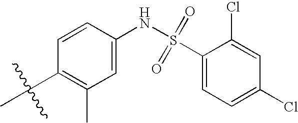 |
| 92 | H | 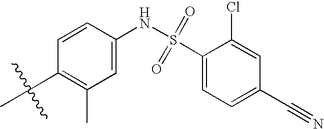 |
| 93 | H | 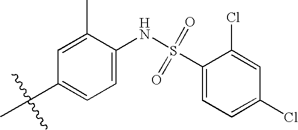 |
| 94 | H | 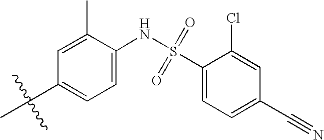 |
| 95 | H | 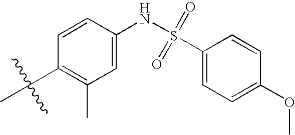 |
| 96 | H | 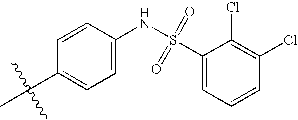 |
| 97 | H | 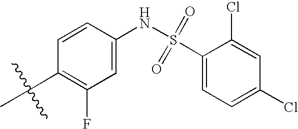 |
| 98 | H | 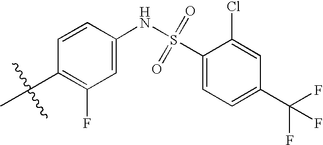 |
| 99 | H | 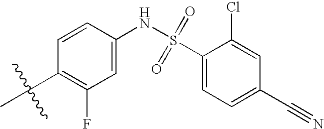 |
| 100 | H | 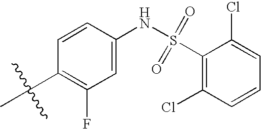 |
| 101 | H | 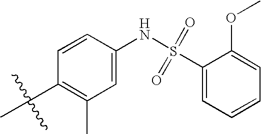 |
| 102 | H | 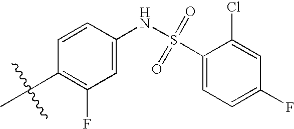 |
| 103 | H | 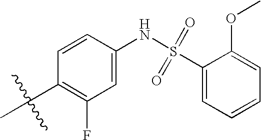 |
| 104 | H | 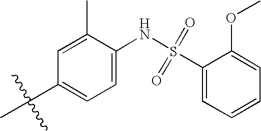 |
| 105 | 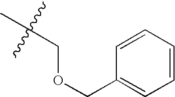 | 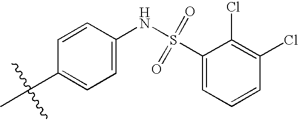 |

or a (preferably pharmaceutically acceptable) salt thereof.

8. The use of a compound of the formula I, or a pharmaceutically acceptable salt thereof, according to claim 1, for the manufacture of a pharmaceutical composition for the treatment of a disease that depends on activity of a protein kinase, especially Tie-2 kinase.

9. The use of a compound of the formula I, or a pharmaceutically acceptable salt thereof, according to claim 1 for the treatment of a disease that depends on activity of a protein kinase, especially Tie-2 kinase.

10. A pharmaceutical formulation, comprising a compound of the formula I, or a pharmaceutically acceptable salt thereof, according to claim 1 and at least one pharmaceutically acceptable carrier material.

11. A method of treatment of a disease that depends on activity of a kinase, especially Tie-2 kinase, comprising administering to a warm-blooded animal, especially a human, in need of such treatment a pharmaceutically effective amount of a compound of the formula I, or a pharmaceutically acceptable salt thereof, according to claim 1.

12. A compound of the formula I, or a pharmaceutically acceptable salt thereof, according to claim 1 for use in the diagnostic or therapeutic treatment of an animal or human body, especially for treatment of a kinase dependent disease, preferably a disease that depends on Tie-2.

13. A process or method for the manufacture o a compound of the formula I according to claim 1, comprising reacting
a) a compound of the formula II, 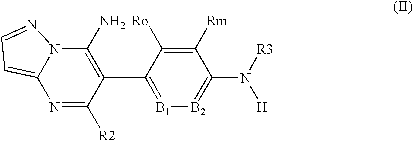
wherein R2, R3, B₁, B₂, Ro and Rm are as defined for a compound of the formula I, with an acid of the formula III,
R1-OH (III)
or a reactive derivative thereof, wherein R1 is as defined for a compound of the formula I, or
b) a nitrile of the formula IV, 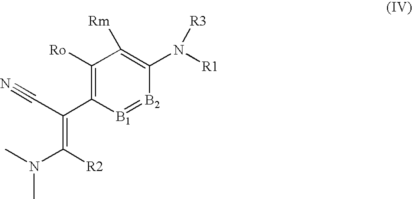
wherein R1, R2, R3, B₁, B₂, Ro and Rm are as defined for a compound of the formula I, with 3-aminopyrazole;
and, if desired, transforming a compound of formula I into a different compound of formula I, transforming a salt of an obtainable compound of formula I into the free compound or a different salt, transforming an obtainable free compound of formula I into a salt thereof, and/or separating an obtainable mixture of isomers of a compound of formula I into individual isomers.
